(19) United States
(12) Patent Application Publication
(10) Pub. No.: US 2014/0287460 A1
(43) Pub. Date: Sep. 25, 2014

(54) PRODUCTION OF RECOMBINANT PROTEIN IN INSECT CELLS USING A BACULOVIRUS EXPRESSION SYSTEM

(71) Applicant: Daniel J. FITZGERALD, Geneva (CH)

(72) Inventor: Daniel J. FITZGERALD, Geneva (CH)

(73) Assignee: GBIOTECH SARL, Plan-les-Ouates, Geneva (CH)

(21) Appl. No.: 14/234,777

(22) PCT Filed: Jul. 26, 2012
(86) PCT No.: PCT/EP2012/064745

§ 371 Date: Mar. 31, 2014

Related U.S. Application Data
(60) Provisional application No. 61/512,039, filed on Jul. 27, 2011.

(51) Int. Cl. C12N 15/86; C12P 21/00

(52) U.S. Cl. CPC C12N 15/86; C12P 21/00
USPC 435/69.1; 435/320.1; 435/348

(57) ABSTRACT

The present invention describes a novel method for recombinant protein production using a baculovirus protein expression system in insect cells, wherein baculovirus virion production is suppressed during recombinant protein production. The novel expression system produces reduced levels of baculovirus virions during recombinant protein production phase, thereby reducing the need for purification of the recombinant protein from the contaminating virus. The novel baculovirus expression system may further suppress expression of recombinant protein by baculovirus-infected cells during virus amplification prior to induction, thereby reducing selection pressure on the expression cassette for the recombinant protein.

### FIELD OF THE INVENTION

The invention relates to an improved method for the production of recombinant protein in insect cells using a baculovirus expression system, to said baculovirus expression system and to components and kits related thereto. The inventive method provides certain advantages, including that recombinant protein can be harvested with reduced amounts of contaminating baculovirus virions, that both amplification/scale-up of baculovirus and production of recombinant protein can be conducted using the same clonal insect cell line, and that standard insect cell lines commonly used for production of recombinant protein using baculovirus can be compatible with the method.

### BACKGROUND OF THE INVENTION

### Recombinant Protein Expression

A vast number of expression systems are used to produce recombinant proteins, ranging from cell free systems to cell based systems. Presently, due to technical limitations associated with cell free expression systems, cell based systems are more commonly used for recombinant protein expression. Cell based expression systems include those utilizing bacteria, yeast, insect cells or mammalian cells as hosts.

One commonly used recombinant protein expression system is the baculovirus insect cell expression system. Baculoviruses have very species-specific tropisms among invertebrate cells and are not known to replicate in mammalian or other vertebrate animal cells. For this reason of safety, and for a number of other reasons including high recombinant protein yield, and lower cost relative to mammalian expression systems, the baculovirus expression system has become one of the most widely used eukaryotic systems for production of recombinant proteins (Nature Biotechnology 23, 567-575). The baculovirus Autographa californica multicapsid nuclear polyhedrosis virus (AcMNPV) is by far the most common vehicle in this system.

The baculoviruses are a family of large, rod-shaped, enveloped viruses that contain circular double-stranded DNA genomes ranging from 80-180 kilo base pairs (kbp). Baculovirus infection of host cells can be divided to three distinct phases: “early” (0-6 h post-infection (p.i.)), “late” (6-24 h p.i.) and “very late” (18-24 to 72 h p.i.). The baculovirus “very late” promoters display extremely high rates of transcription relative to host cell promoters and early or late baculovirus promoters. Therefore, the basic idea behind prior-art baculovirus expression of recombinant proteins in insect cells is that the DNA sequence coding for a recombinant protein of interest is shuttled into the baculovirus genome under the control of such a “very late” promoter, which results in high levels of expression of the recombinant protein during (very late stages of) infection of the cultured (host) insect cell.

A number of technological improvements have eliminated the original tedious procedures required to create and culture recombinant baculoviruses. Modern baculovirus expression systems allow for recombinant genes to be shuttled into the baculovirus genome through recombination of baculovirus DNA and recombinant gene-containing plasmids in insect cells, in bacteria, or in vitro. Using any of these methods, sufficient recombinant protein-encoding viruses are typically generated to infect insect cell culture volumes in the order of a few milliliters. However, for baculovirus-based applications such as vaccine production, or adeno-associated virus (AAV) production, tens, hundreds, or even thousands of liters of insect cell culture are required to generate the desired levels of recombinant protein. One example requiring large scale baculovirus expression is the virus like particle (VLP) based cervical cancer vaccine CERVARIX™ from GlaxoSmithKline. Scale-up for large scale baculovirus expression requires an exponential increase in the number of recombinant viruses from the starting point of small numbers of recombinant viruses, such as from a small pool on the order of millions of virus particles. This requires intact virus replication as well as production of infectious virions to spread the infection. Scale-up is typically carried out by allowing the recombinant viruses to replicate in sequentially larger culture volumes and harvesting the resultant virus particles for generation of a master virus seed stock. Subsequent recombinant protein production using this virus seed stock is then typically carried out in a separate culture setup. During the scale-up phase, the generation of replication competent, infectious baculovirus particles is required. However, during protein production phase, if sufficient baculovirus is used to infect the entire insect cell culture, co-production of new infectious virions to spread the infection throughout the culture used for production of recombinant protein is not required, and in certain applications is not desired. Indeed, the co-production of baculovirus virions together with the recombinant protein of interest can represent a serious drawback of baculovirus expression systems, as it can be both difficult and costly to separate the co-produced baculovirus from the recombinant protein. This is a particular problem in the production of recombinant proteins used in therapeutic or prophylactic applications, since AcMNPV is known to have immunoadjuvant properties, such as inducing anti-viral cytokine production (J Immunol 171, 1133-1139; Journal of Virology 73, 9944-9951) and recognition by Toll-like receptors (Clin. Vaccine Immunol. 15, 376-378). Hence, it is desirable to avoid contamination of such a recombinant protein with both, budded virions (BVs) and occlusion-derived virions (ODVs).

Still another problem observed with baculovirus-based recombinant protein production is that the yield at small scale (e.g. milliliters) is typically observed to be up to several fold higher than at tens or hundreds of liter scale. Specifically, yield of protein produced per volume cell culture decreases stepwise per virus generation even when very strict precautions are followed (see Baculovirus Expression Vectors, O'Reilly et al. Oxford University Press, New York, 1994). One potential cause for this decrease in production yield during scale-up is that defective interfering (DI) viruses exponentially build up as baculoviruses undergo the multiple cycles of replication required to scale-up protein production (Biotech. Letters 13, 483-488; Virology 283, 132-138; Journal of General Virology, 84, 2669-2678). DI viruses are mutant viruses, often carrying large (up to 40%) deletions of their DNA (Virology 283, 132-138), including deletions of the recombinant protein expression cassette. The accumulation of DI viruses during scale-up can be avoided, through careful monitoring of the relative ratio of infections particles to insect cells during scale-up (see Baculovirus Expression Vectors, O'Reilly et al. Oxford University Press, New York, 1994).

Independent of DI virus accumulation, a second phenomenon that can cause drop in protein production yield during scale-up is the accumulation of mutations in the recombinant protein DNA expression cassette.

In prior art baculovirus expression systems, recombinant protein are constitutively transcribed and translated at very high levels and therefore require a significant fraction of the cell's total metabolic capacity. In addition, recombinant proteins are not essential for replication of the virus. This results in a high selection pressure for generation of mutant viruses, which do not transcribe/translate the recombinant protein, such as those that no longer carry the non-essential recombinant protein expression cassette. It is expected that scaling up baculovirus expression to larger and larger culture volumes will result in a progressive accumulation of viruses that do not carry a functional recombinant protein (DNA) expression cassette (Journal of General Virology, 84, 2669-2678; Nature Methods 3, 1021-1032). This is not too surprising, given the well documented phenomenon of plasmid loss experiments, which demonstrate that non-essential DNAs are very rapidly removed from a wide-range of cell types in the absence of selection markers to maintain them (Plasmid 36, 161-167). In the case of specific elimination or other forms of mutation of the recombinant protein DNA expression cassette during baculovirus scale up, particular DNA sequences in or around the recombinant protein DNA expression cassette are expected to be hotspots for elimination/mutation.

There are a number of commercial systems available for expressing recombinant proteins using baculovirus, including flashBAC™ (Oxford Expression Technologies EP 1 144 666), BackPack™ (BD Biosciences Clontech), BacVector® 1000/2000/3000 (Novagen®), BAC-TO-BAC® (Invitrogen™ U.S. Pat. No. 5,348,886), and BaculoDirect™ (Invitrogen™). All of these systems are based on the principle of expressing recombinant proteins by placing them under the control of the very late baculovirus promoters polh or p10. None of these systems allow for inducible control of protein expression, particularly, for repression of recombinant protein expression during virus amplification as part of the scale up phase, or for repression of baculovirus production during the protein production phase.

One baculovirus-based technology which does allow inducible control of protein production is the “BacMam” technology which uses baculoviruses as vehicles to deliver and inducibly express recombinant proteins in mammalian cells (Nature Biotechnol. 23: 567-575). This system does not allow inducible expression of recombinant proteins in insect cells, and does not use the heavily transcribed baculovirus very late promoters for recombinant protein expression; rather it uses mammalian promoters, because baculovirus very late promoters do not fire in mammalian cells. Corresponding approaches that use baculovirus technology to deliver an expression system into mammalian cells, for inducible expression of a recombinant protein in mammalian cells, have also been disclosed by McCormick et al (J Gen Virol, 2002; 83: 383-394 and J Gen Virol, 2004; 85: 429-439).

Aslanidi et al (PNAS, 2009; 106: 5059-5064) describe an inducible system for production of virus vectors in insect cells. This system uses baculovirus as a gene transfer vector to provide, upon infection of insect cells by baculovirus, the baculovirus-encoded transcription factors that are required to carry out recombinant protein expression. This system does not provide for suppression of baculovirus virion production during the recombinant protein production phase, or for suppression of recombinant protein expression during the scale up phase.

Wu et al., (Journal of Biotechnology, 80; 75-83) created an effective, plasmid based tetracycline regulatory expression system (TRES) for use in insect cells. However, high background activation of a minimal human cytomegalovirus immediate-early (CMVm) promoter by the viral polyhedrin upstream (pu) sequence precluded their parallel development, as reported in this publication, of a similarly effective inducible baculovirus expression system for insect cells. In a follow-up study by the same group, deletion of the pu element resulted in an effective inducible (TRES) baculovirus system for insect cells (Biotechnol. Prog., 24; 1232-1240). Using the system they demonstrated that inducible overexpression of Lef-2 stimulates polh transcription and its associated recombinant protein expression. However, their system did not demonstrate the capability to down-regulate very late promoter-driven (polh-driven) transcription and its associated protein expression, rather only (further) up-regulation, resulting in a recombinant protein expression from the very late polh promoter exceeding wild type levels. This system therefore appears unable to overcome the problem of high very late promoter driven (essentially constitutive) recombinant protein expression during up-scaling of baculovirus expression and the associated high selective pressure against the expression cassette encoding the recombinant protein, that can resulting in elimination and/or mutation of this cassette. This system does not provide for repression of baculovirus production during the protein production phase.

U.S. Pat. No. 5,939,285 describes the use of a retinoic acid response element (RARE) in a baculovirus promoter (such as polh or p10) to regulate expression of a recombinant protein in insect cells in the presence of a hormone receptor expressed by a gene encoding the same. Such regulation is brought about by varying (between different expression systems) the position and particular DNA arrangement of the RARE/promoter construct relative to the open reading frame encoding the recombinant protein. Such regulation is not brought about by subjecting or exposing any given expression system to a change in conditions. This system does not provide for an inducible baculovirus system that allows repression of baculovirus production during the protein production phase

Dai et al., (Protein Expression and Purification, 2005; 42; 236-245) created an effective, plasmid-based ecdysone receptor transcriptional induction system for use in insect cells. However, the authors did not demonstrate functionality of a comparable inducible system using baculovirus as a vector.

Nicholson et al., (Molecular Therapy, 2005, 11(4), 2005) and Starkey et al., (Journal of General Virology, 2009; 90, 115-126) both describe RNA interference expression cassettes present on baculovirus DNA, but the RNAi expression cassettes are not inducible.

Kanginakudru et al., (Insect Molecular Biology, 2007, 16(5), 635-644), Valdes et al., (Journal of Biological Chemistry, 2003, 278(21), 19317-19324) and Flores-Jasso et al., (Virus Research, 2004, 102, 75-84) describe using RNA interference to inhibit baculovirus replication, but none of these studies describe using inducible transcription of the RNA interference effector to inhibit baculovirus replication.

A system for baculoviral production of virion-free recombinant protein in insect cells has been provided by Marek and coworkers (Biotechnology and Bioengineering 108, 1056-1067; 2010) who describe the use of a specifically engineered baculovirus carrying a deletion of the structural protein VP80 thereby preventing the formation of budded virus as well as occlusion-derived virus. This vp80-deleted virus lacks an infectious phenotype and does not propagate in conventional insect cells. Hence, during scale up using the system of Marek and coworkers the vp80-deleted baculovirus is amplified in specially engineered insect cells which constitutively express VP80 to complement the vp80-deletion mutation. During subsequent recombinant protein production using the system of Marek and coworkers, the amplified (vp80-delection mutant) baculovirus are then transferred to a conventional insect cell line, which is then used for production of the desired recombinant protein. For any large scale protein production process the use of two cell lines rather than one may be disadvantageous. In addition, any newly developed cell line to be used for production of therapeutic proteins, such as Marek and coworkers' VP80 expressing cell line, is required by major regulatory agencies to pass through all stages of regulatory acceptance. Therefore, in the absence of significant advantages in using novel cell lines (and/or baculovirus strains), the use of conventional or regulatory-accepted cell lines for both scale up and recombinant production of therapeutic proteins is preferable to the use of novel cell lines.

A similar vp80 deletion baculovirus/VP80 complementing insect cell line system is further disclosed by WO 2011/020710. In addition, in a second approach it is further proposed that insect cells may be engineered to express a dsRNA which targets an essential baculovirus capsid gene such as vp80, by RNA-mediated silencing or by RNA interference. The inventors propose that such a method could be advantageous over their vp80 deletion baculovirus/VP80 complementing insect cell line because conventional insect cells could be used for process scale up, while the dsRNA expressing cell lines would only be necessary during recombinant protein production to inhibit co-production of baculovirus particles. However, as with the approach using vp80 deletion baculovirus/VP80 complementing insect cell line, this second approach uses a biopharmaceutical-producing insect cell line (see claim 1 of WO 2011/020710) which is a distinct from a second baculovirus producing cell line (see claim 4 of WO 2011/020710). Furthermore, as with the VP80 complementing cell line method, the dsRNA expressing cell line would be considered by major regulatory agencies as a new cell line and would likely be required to start and pass through all stages of regulatory acceptance.

Hence there is a need for an improved or alternative method for the production of a recombinant protein with reduced contaminating baculovirus virions, where both the scale up and recombinant protein production phases are carried out in the same cell line, and preferably is a conventional insect cell line. There is further a need for a method where additionally the expression of recombinant protein can be repressed during amplification of baculovirus/scale up of the culture volume.

Hence, it is an objective of the invention to provide a method for the production of a recombinant protein using a baculovirus system in insect cells, wherein the production of baculovirus virions is provided during scale up phase, but suppressed during recombinant protein production phase. It is a further object of the invention to provide a method for production of a recombinant protein using a baculovirus system in insect cells, wherein the production of baculovirus virions is allowed while simultaneously recombinant protein production is suppressed during scale up phase and wherein the production of baculovirus virions is suppressed while simultaneously recombinant protein production is allowed during recombinant protein production phase, allowing more effective up-scaling and higher overall recombinant protein expression with reduced contaminating baculovirus virions. It is another object of the invention to provide a baculovirus expression system and components thereof useful in the method.

### SUMMARY OF THE INVENTION

One or more of the above objects are solved by the methods, baculovirus expression system, components and kits as described and claimed herein. By virtue of the methods and baculovirus expression system of the present invention, it is now possible to provide for virus amplification and production during up-scaling of the infected insect cell culture and to suppress expression of at least one gene product essential for baculovirus virion assembly, thereby suppressing baculovirus virion production during recombinant protein production phase. As a result, the recombinant protein produced comprises reduced amounts of baculovirus virions. The inducible baculovirus expression of the present invention also provides for the repression of recombinant protein expression during amplification of the virus and up-scaling of infected insect cell culture, thereby reducing selection pressure on the virus and the cell for elimination and/or mutation of the expression cassette encoding the recombinant protein. The result is a higher ratio of recombinant protein producing virus to non-recombinant protein producing virus relative to currently available baculovirus expression systems, and/or a system that is easier to use, up-scale or to use for the production of biopharmaceutical products that are subjected to tight and close regulatory restriction and approval. Consequently, after inducing recombinant protein expression and suppressing expression of at least one gene essential for baculovirus virion assembly, higher concentrations or larger amounts of recombinant protein per volume of insect cell culture can be achieved with reduced concentrations or amounts of contaminating baculovirus virions. The method for producing recombinant protein using the baculovirus expression system of the present invention is broadly applicable and allows for production of high yields of recombinant protein even in large industrial-scale cell cultures involving many virus passages.

Thus in one aspect the invention relates to a method for the production of a recombinant protein in insect cells, comprising the steps of (a) providing insect cells comprising a baculovirus expression system containing a nucleotide sequence encoding said recombinant protein; (b) maintaining during a scale up phase said insect cells of step (a) under a first condition such that said baculovirus replicates to produce infectious baculovirus virions; (c) maintaining during a recombinant protein production phase said insect cells of step (b) under a second condition such that production of baculovirus virions is suppressed; and (d) harvesting said recombinant protein expressed by said baculovirus expression system, wherein the baculovirus expression system comprises an inducible expression control system that suppresses expression of at least one gene product essential for baculovirus virion assembly under said second condition during production of said recombinant protein; and allows expression of said at least one gene product essential for baculovirus virion assembly under said first condition.

Optionally, recombinant protein expression can be suppressed during virus amplification and scale up phase in said insect cells.

In another aspect, the invention relates to a baculovirus expression system as defined herein, for example one for use in a method of the present invention.

In other aspects, the invention relates to certain components of said baculovirus expression system, or components, compositions or kits for constructing or using such baculovirus expression system in a method of the present invention, including components such as certain baculovirus transfer vectors, composite baculovirus DNA, insect cells, vectors and recombinant nucleic acids.

Generally, and by way of brief description, the present invention describes a novel baculovirus protein expression system in insect cells, wherein the baculovirus virion production can be suppressed during recombinant protein production phase using an baculovirus expression system. By the methods using a baculovirus expression system of the invention, expression of a gene product essential for baculovirus virion production can be suppressed, thereby inhibiting virion assembly following scale up phase in the presence or absence of an inducing molecule or by a change in environmental conditions. Using the baculovirus expression systems of the invention, in certain embodiments also expression of recombinant protein can be suppressed during scale up phase, thereby reducing selection pressure on the expression cassette for the recombinant protein, and then expression of recombinant protein can be selectively induced during recombinant protein production phase, thereby improving the yield of protein expression derived from the described methodology and reducing time and effort for recombinant protein purification after cell harvest, particularly for recombinant proteins used as biopharmaceuticals.

### BRIEF DESCRIPTION OF THE DRAWINGS

FIG. 1: Repressor-based induction of RNAi effector transcription to modulate recombinant baculovirus production. The targeting of the RNAi effector molecule (RNA, triangle) from Y to the RNA product of Z is indicated. The binding of repressor protein (Rep, circle)—a controllable transcriptional modulator protein and encoded by expression cassette X—to its DNA binding site in Y— the transcriptional repressor response element (light grey dashed rectangle), is similarly indicated. Promoters of cassettes X, Y, and Z are represented by dark rectangles. The “L/VL” promoter in Y refers to any promoter that functions as a late or very late baculovirus promoter. Binding of the repressor protein from X to its response element in Y reversibly represses RNAi effector molecule transcription as indicated by the thick arrow (left). RNAi effector molecule production from cassette Y regulates the levels of recombinant baculovirus production as indicated by the thick arrow (right), by reversibly modulating the levels of available essential baculovirus capsid protein. In all such general embodiments of the invention shown schematically in FIGS. 1 to 3, it being understood by the person of ordinary skill, upon disclosure of the invention, that the transcriptional modulator protein encoded by expression cassette X is a controllable transcriptional modulator protein, and such controllability is used to induce transcription, or to initially repress, and then induce upon removal of such repression.

FIG. 2: Activator-based induction of RNAi effector transcription to modulate recombinant baculovirus production. The targeting of the RNAi effector molecule (RNA, triangle) from Y to the RNA product of Z is indicated. The binding of activator protein (Act, circle)—a controllable transcriptional modulator protein and encoded by expression cassette X—to its DNA binding site in Y— the transcriptional activator response element (light grey dashed rectangle), is similarly indicated. Promoters of cassettes X, Y, and Z are represented by dark rectangles. The “minimal” promoter in Y refers to a promoter that displays minimal constitutive activity. Binding of the activator protein from X to its response element in Y reversibly activates RNAi effector molecule transcription as indicated by the thick arrow (left). RNAi effector molecule production from cassette Y regulates the levels of recombinant baculovirus production as indicated by the thick arrow (right), by reversibly modulating the levels of available essential baculovirus capsid protein.

FIG. 3: Repressor-based direct induction of recombinant baculovirus production. The binding of repressor protein—a controllable transcriptional modulator protein and encoded by expression cassette X—(Rep, circle) to its DNA binding site—the transcriptional repressor response element in Z, is indicated. Binding of the repressor to cassette Z controls recombinant baculovirus production as indicated by the thick arrows, by reversibly modulating the levels of available essential baculovirus capsid protein.

FIG. 4: Example 1. Repressor-based induction of RNAi transcription to modulate recombinant baculovirus production. The targeting of the shRNA RNAi effector molecule (shRNA, triangle) from Y to the RNA product of Z, the essential capsid protein VP80, is indicated. The binding of the tetracycline repressor protein from cassette X (TetR, circle) to its DNA binding site in Y (light grey dashed rectangle), is similarly indicated. Promoters of cassettes X (pe38 promoter), Y, (ORF-54 promoter) and Z (vp80 promoter) are represented by dark rectangles. Binding of TetR from X to its response element in Y reversibly represses the shRNA RNAi effector molecule transcription as indicated by the thick arrow (left). shRNA RNAi effector molecule production from cassette Y regulates the levels of recombinant baculovirus production as indicated by the thick arrow (right), by reversibly modulating the levels of available essential baculovirus capsid protein VP80.

FIG. 5: Example 1. Generation of composite baculovirus DNA containing expression cassettes X, Y, and Z as described in FIG. 4. Baculovirus DNA bMON14272 (left) contains expression cassette Z encoding the native VP80 protein. The transfer vector pE1 (right) contains expression cassettes X and Y encoding the tet repressor protein and shRNA, respectively. In transfer vector pE1, direction of transcription 5′ to 3′ for expression cassettes X and Y is indicated by arrows. Promoter sequences pe38 for tetR and ORF-54 for shRNA are indicated as are the DNA binding sites for tet repressor protein. Fusion of the transfer vector pE1 and bMON14272 is carried out by Tn7 transposition (indicated by the dashed cross).

FIG. 6: Example 2. Activator-based induction of RNAi transcription to modulate recombinant baculovirus production. The targeting of the shRNA RNAi effector molecule (shRNA, triangle) from Y to the RNA product of Z, the essential capsid protein VP80, is indicated. The binding of the ecdysone activator protein from cassette X (EcR, circle) to its DNA binding site in Y (light grey dashed rectangle), is similarly indicated. Promoters of cassettes X (pe38 promoter), Y, (ie2 promoter) and Z (vp80 promoter) are represented by dark rectangles. Binding of EcR from X to its response element in Y reversibly activates the shRNA RNAi effector molecule transcription as indicated by the thick arrow (left). shRNA RNAi effector molecule production from cassette Y regulates the levels of recombinant baculovirus production as indicated by the thick arrow (right), by reversibly modulating the levels of available essential baculovirus capsid protein VP80.

FIG. 7: Example 2. A) Generation of composite baculovirus DNA containing expression cassettes X, Y, and Z as described in FIG. 6. Baculovirus DNA bMON14272 (left) contains expression cassette Z encoding native vp80 protein. The transfer vector pE2 (right) contains expression cassettes X and Y encoding the ecdysone activator protein and shRNA, respectively. In transfer vector pE2, direction of transcription 5′ to 3′ for expression cassettes X and Y is indicated by arrows. Promoter sequences pe38 for ecdysone activator and ie-2 minimal for shRNA are indicated as are the DNA binding sites for ecdysone activator protein. Fusion of the transfer vector pE2 and bMON14272 is carried out by Tn7 transposition (indicated by the dashed cross). B) Comparison of budded infectious virus production from inducible bMON14272-pE2 (left) and non-inducible bMON14272-pDual (right) in the presence and absence of inducer molecule RG-102240. Baculovirus infected SF21 cells are incubated for 72 hours in the presence, or absence of 0.5 μM RG-102240 (indicated by + or −, bottom). A comparable quantity of cell supernatant is compared for infectious virus titre as measured by an end point dilution assay. S.E.M of three measurements is indicated by dark bars on the top of the graphs.

FIG. 8: Example 3. Repressor-based direct induction of recombinant baculovirus production. The binding of the Tet-On repressor protein—a controllable transcriptional modulator protein and encoded by expression cassette X—(Tet-On, circle) to its DNA binding site—the transcriptional repressor response element in Z, is indicated. Z encodes the essential capsid protein VP80. Promoters of cassettes X (pe38 promoter), and Z (vp80 promoter) are represented by dark rectangles. Binding of Tet-On to cassette Z controls recombinant baculovirus production as indicated by the thick arrows, by reversibly increasing the levels of available essential baculovirus capsid protein.

FIG. 9: Example 3. Generation of composite baculovirus DNA containing expression cassettes X, and Z as described in FIG. 8. Baculovirus DNA modified bMON14272 (left) contains expression cassette Z encoding modified vp80 expression cassette. vp80 is modified through ET recombination to fuse the indicated DNA fragment with bMON14272 (upper left, dashed lines indicate recombination reaction). The transfer vector pE3 (right) contains expression cassettes X encoding the Tet-On repressor protein. In transfer vector pE3, direction of transcription 5′ to 3′ for expression cassette X is indicated by an arrow. The pe38 promoter sequence for Tet-On is indicated as is its DNA binding site in the vp80 promoter (upper left, small dashed rectangle). Fusion of the transfer vector pE3 and modified bMON14272 is carried out by Tn7 transposition (indicated by the dashed cross).

FIG. 10: Example 4. Repressor-based induction of RNAi transcription to modulate recombinant baculovirus production and recombinant protein production: The targeting of the shRNA RNAi effector molecule (shRNA, triangle) from Y to the RNA product of Z, the essential capsid protein VP80, is indicated. The binding of the tetracycline repressor protein from cassette X/A (TetR, circle) to its DNA binding sites in Y and C (light grey dashed rectangles), is similarly indicated. Promoters of cassettes X/A (pe38 promoter), Y, (ORF-54 promoter), Z (vp80 promoter), and C (polh promoter) are represented by dark rectangles. Binding of TetR from X/A to its response element in Y reversibly represses the shRNA RNAi effector molecule transcription, while simultaneous binding of TetR to its response element in C represses EGFP recombinant protein production as indicated by the thick arrows (left). shRNA RNAi effector molecule production from cassette Y regulates the levels of recombinant baculovirus production as indicated by the thick arrow (right), by reversibly modulating the levels of available essential baculovirus capsid protein VP80. Similarly, EGFP levels are regulated by the reversible binding of TetR to C as indicated by the thick arrow (right).

FIG. 11: Example 4. Generation of composite baculovirus DNA containing expression cassettes X/A, Y, Z, B, and C as described in FIG. 10. Baculovirus DNA bMON14272 (left) contains expression cassettes Z encoding the native VP80 protein, and B encoding the native vlf-1 protein. The transfer vector pE4 (middle) contains expression cassettes X/A encoding the tet repressor protein, Y encoding the shRNA, and C encoding EGFP. In transfer vector pE4, direction of transcription 5′ to 3′ for expression cassettes X/A and Y and C is indicated by arrows. Promoters of cassettes X/A (pe38 promoter), Y (ORF-54 promoter), Z (vp80 promoter), and C (polh promoter) are represented by dark rectangles. Fusion of the transfer vector pE4 and bMON14272 is carried out by Tn7 transposition (indicated by the dashed cross). Alternatively fusion of the control transfer vector pE4.1 and bMON14272 is also carried out by Tn7 transposition (not indicated).

FIG. 12: Example 5. Repressor-based direct induction of recombinant baculovirus production and recombinant protein production. The binding of the Lac repressor protein—a controllable transcriptional modulator protein and encoded by expression cassette X′—(LacR, circle) to its DNA binding site—the transcriptional repressor response element in Z, is indicated. Z encodes the essential capsid protein VP80. The binding of the tetracycline repressor protein from cassette X (TetR, circle) to its DNA binding sites in X′ and C (light grey dashed rectangles), is similarly indicated. Promoters of cassettes X′ (ORF-54 promoter), Z (vp80 promoter) X (pe38 promoter) and C (polh promoter) are represented by dark rectangles. Binding of LacR to cassette Z controls recombinant baculovirus virion production as indicated by the thick arrows (right), by reversibly repressing the levels of available essential baculovirus capsid protein VP80. Binding of TetR from X to its response element in X′ reversibly represses LacR production, while simultaneous binding of TetR to its response element in C reversibly represses EGFP recombinant protein production as indicated by the thick arrows (left).

FIG. 13: Example 5. Generation of composite baculovirus DNA containing expression cassettes X′, Z, X, B, and C as described in FIG. 12. Baculovirus DNA modified bMON14272 (left) contains expression cassette Z encoding modified vp80 expression cassette, and B, encoding native vlf-1 protein. vp80 is modified through ET recombination to fuse the indicated DNA fragment with bMON14272 (upper left, dashed lines indicate recombination reaction). The transfer vector pE5 (right) contains expression cassettes X′ encoding the Lac repressor protein, X encoding the tetracycline repressor protein, and C, encoding EGFP. In modified bMON14272 and transfer vector pE5, direction of transcription 5′ to 3′ for all expression cassettes is indicated by arrows. Promoters of cassettes X′ (ORF-54 promoter), Z (vp80 promoter), X (pe38 promoter), and C (polh promoter) are represented by dark rectangles. DNA binding sites for LacR, TetR, and vlf-1 are represented by dashed grey rectangles. Fusion of the transfer vector pE5 and modified bMON14272 is carried out by Tn7 transposition (indicated by the dashed cross).

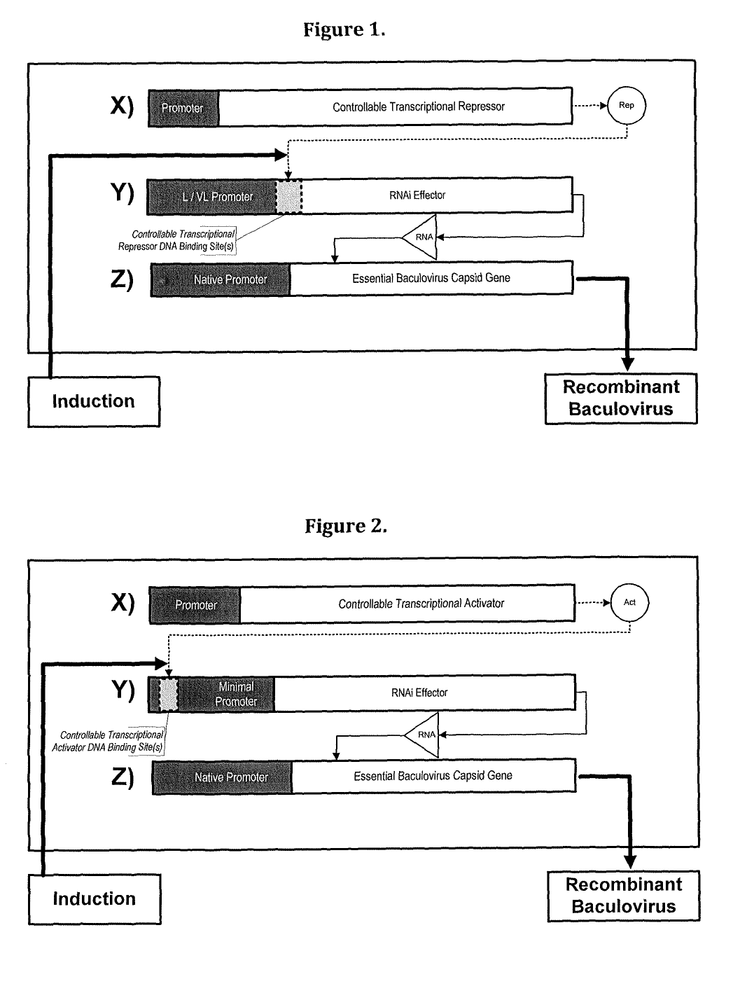

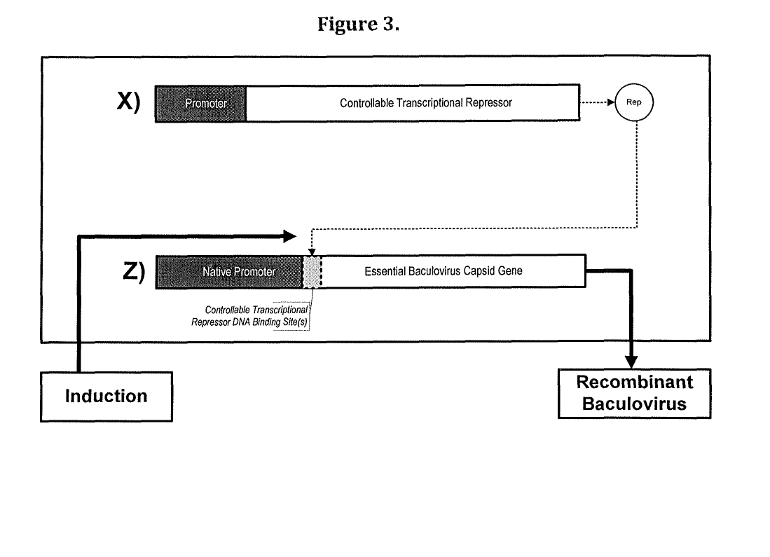

### DETAILED DESCRIPTION OF THE INVENTION

Provided herein is a method for the production of a recombinant protein using a baculovirus expression system, wherein expression of at least one gene product essential for baculovirus virion assembly is repressed during recombinant protein production phase, in insect cells. Optionally, recombinant protein expression can be repressed during virus amplification and scale up phase in insect cells.

By virtue of the method of the invention, it is possible to produce recombinant protein in insect cells with reduced or no contamination of baculovirus virions, and optionally further to passage virus with higher levels of non-defective virus (ie those able to express recombinant protein) relative to what is achieved by currently available methods.

In one aspect, the invention relates to a method for the production of a recombinant protein in insect cells, comprising the steps of (a) providing insect cells comprising a baculovirus expression system capable of expressing a nucleotide sequence encoding said recombinant protein; (b) maintaining during a scale up phase said insect cells of step (a) under a first condition such that said baculovirus replicates to produce infectious baculovirus virions; (c) maintaining during a recombinant protein production phase said insect cells of step (b) under a second condition such that production of baculovirus virions is repressed; and (d) harvesting said recombinant protein expressed by said baculovirus expression system, wherein the baculovirus expression system comprises an inducible expression control system that suppresses expression of at least one gene product essential for baculovirus virion assembly under said second condition during production of said recombinant protein; and allows expression of said at least one gene product essential for baculovirus virion assembly under said first condition.

Preferably, the at least one gene product essential for baculovirus virion assembly is selected from the group consisting of vp80, vp39, vp1054, gp64, p74, p24 and p6.9. More preferably at least one gene product essential for baculovirus virion assembly is a vp80 baculovirus capsid protein (Seq ID No: 8). In certain embodiments, such gene product essential for baculovirus virion assembly is under the control of its native promoter. Also preferably, the insect cell is a standard cell line selected from the group consisting of SF9, SF21, High Five™ Cells (BTI-TN-5B1-4) and Mimic™ Sf9 insect cells.

In one embodiment of such invention the baculovirus expression system comprises at least one expression cassette X comprising a promoter and an open reading frame coding for a controllable transcriptional modulator protein; at least one expression cassette Y comprising a promoter and a nucleotide sequence coding for an RNA-mediated silencing and/or RNA interference (RNAi) effector targeting said at least one gene product essential for baculovirus virion assembly; at least one expression cassette Z comprising a promoter and an open reading frame coding for said gene product essential for baculovirus virion assembly; and at least one transcriptional modulator response element in expression cassette Y, wherein said controllable transcriptional modulator protein reversibly interacts with said transcriptional modulator response element(s) in said first condition, and interacts differently in said second condition, thereby modulating the transcription of expression cassette Y. In one preferred embodiment the at least one gene product essential for baculovirus virion assembly in expression cassette Z is under the control of its native promoter, preferably it is a vp80 baculovirus capsid protein (Seq ID No: 8) under the control of its native late promoter (Seq ID No: 9). As will be appreciated by the person of ordinary skill following the disclosure of the present invention, the RNAi effector encoded by expression cassette Y, when transcribed may then be processed by one or more cellular enzymes such as Dicer (Curr Top Microbiol Immunol. 320, 77-97), before binding to and leading to post-transcriptional depletion of the mRNA transcript of expression cassette Z, i.e. depletion of the transcript encoding for a gene product essential for baculovirus virion assembly, thereby suppressing the expression of said gene product.

In certain such embodiments the RNAi effector is a short interfering RNA (siRNA), a short hairpin RNA (shRNA), a long hairpin RNA (IhRNA) or a polycystronic shRNA, preferably a shRNA or a polycystronic shRNA, and more preferably a shRNA. In certain embodiments one, two or three gene product(s) essential for baculovirus virion assembly is/are targeted. Preferably two or more gene products essential for baculovirus virion assembly are targeted. Two or more gene product(s) essential for baculovirus virion assembly may be targeted by one RNAi effector encoded by one expression cassette Y, targeting two or more gene product(s) essential for baculovirus virion assembly, or alternatively by two or more RNAi effectors encoded by different expression cassettes Y, each targeting one gene product essential for baculovirus virion assembly. More preferably two or three gene products essential for baculovirus virion assembly are targeted, even more preferably two gene products essential for baculovirus virion assembly are targeted.

Nucleotide sequences suitable to act as an RNAi effector to target a given gene product essential for baculovirus virion can be readily identified and/or selected by the person of the ordinary skill. For example, should an RNAi effector be needed to target a given baculovirus gene, such as the transcripts from vp80, the dsRNA transfection methods of Marek et al., (Biotechnology and Bioengineering 108, 1056-1067), can be used to identify the most effective of such RNAi effector sequences to be comprised in expression cassette Y. Alternatively, the method described in Example 4 can be used to screen for optimal shRNA effector molecules. In a particular embodiment, the nucleotide sequence coding for the RNAi effector is that given in Seq ID No: 3, 4, 5 or 6.

It will be appreciated that according to the invention the transcriptional modulator protein is a transcriptional repressor protein or a transcriptional activator protein. It will be further understood that if the controllable transcriptional modulator protein is a controllable transcriptional repressor protein, the modulator response element is a transcriptional repressor response element. Alternatively, if the transcriptional modulator protein is a transcriptional activator protein, the modulator response element is a transcriptional activator response element capable of activating transcription of expression cassette Y coding for said RNAi effector under the control of a minimal promoter producing low or no RNAi effector in its uninduced state. In preferred embodiments, the controllable transcriptional modulator protein is a controllable transcriptional repressor protein.

Accordingly, in a preferred embodiment the baculovirus expression system comprises: at least one expression cassette X comprising a promoter and an open reading frame coding for a controllable transcriptional repressor protein; at least one expression cassette Y comprising a promoter, a transcriptional repressor response element and a nucleotide sequence coding for an RNAi effector targeting said at least one gene product essential for baculovirus virion assembly; and at least one expression cassette Z comprising a promoter and an open reading frame coding for said gene product essential for baculovirus virion assembly, wherein said first condition maintained during scale up phase is repressive, thereby repressing transcription of said RNAi effector and allowing expression of said gene product essential for baculovirus virion assembly, and said second condition maintained during production phase is non-repressive, thereby inducing transcription of said RNAi effector and suppressing expression of said gene product essential for baculovirus virion assembly; and further wherein the promoter in expression cassette X has an earlier time of onset of gene expression than the promoter in expression cassette Y, the promoter in expression cassette Y has an earlier or simultaneous time of onset of gene expression than the promoter in expression cassette Z. Methods according to this embodiment are also referred to herein as methods according to iVBac1A. Preferably, the promoter in expression cassette Z is the native promoter of said gene product essential for baculovirus virion assembly.

Preferably in such an embodiment, the promoter in expression cassette X is an early promoter, including an early baculoviral promoter such as the pe38 promoter (Seq ID No: 2), or a cellular promoter. The promoter in expression cassette Y preferably has a later time of onset of gene expression compared to the promoter in expression cassette X, such as the late baculovirus promoter ORF-54 (Seq ID No: 7).

In certain of such embodiments the controllable transcriptional repressor protein is selected from the group consisting of TetR, CymR, trpR, MetJ, lac repressor protein and tox repressor protein and preferably the controllable transcriptional repressor protein is TetR (Seq ID No: 1).

In another related embodiment of this aspect of the present invention, the baculovirus expression system comprises at least one expression cassette X comprising a promoter and an open reading frame coding for a controllable transcriptional activator protein; at least one expression cassette Y comprising a minimal promoter, a transcriptional activator response element and a nucleotide sequence coding for an RNAi effector targeting said at least one gene product essential for baculovirus virion assembly; and at least one expression cassette Z comprising a promoter and an open reading frame coding for said gene product essential for baculovirus virion assembly, wherein said first condition maintained during scale up phase is non-activating, thereby repressing transcription of said RNAi effector and allowing expression of said gene product essential for baculovirus virion assembly, and said second condition maintained during recombinant protein production phase is activating, thereby inducing transcription of said RNAi effector and suppressing expression of said gene product essential for baculovirus virion assembly; and further wherein the promoter in expression cassette X has an earlier time of onset of gene expression than the promoter in expression cassette Y, the promoter in expression cassette Y has an earlier or simultaneous time of onset of gene expression than the promoter in expression cassette Z. Methods according to this embodiment are also referred to herein as methods according to iVBac1B. Optionally the promoter in expression cassette Z is the native promoter of said gene product essential for baculovirus virion assembly.

Preferably in such an embodiment, the promoter in expression cassette X is an early promoter, including an early baculoviral promoter such as the pe38 promoter (Seq ID No: 2), or a cellular promoter. The minimal promoter in expression cassette Y preferably has a later time of onset of gene expression compared to the promoter in expression cassette X, such as the ie2 minimal promoter (Protein Expression and Purification 42, 236-245).

In certain of such embodiments the controllable transcriptional activator protein is selected from the group consisting of metallotheionein (MT), AMT1, glucocorticoid receptor protein (GC), estrogen receptor, ecdysone receptor, AlcR, Tet-On, Tet-Off, CAP, AP-1, WRKY1, WRKY2 and WRKY3. Preferably the controllable transcriptional activator protein is a modified ecdysone receptor protein (EcR).

In particular embodiments of the method of the present invention, additionally under said first condition the expression of said recombinant protein is suppressed. Preferably in such embodiments, said first condition suppresses expression of said recombinant protein in addition to allowing production of said baculovirus virions; and said second condition allows expression of said recombinant protein in addition to suppressing production of said baculovirus virions.

In one such embodiment of the invention the baculovirus expression system comprises at least one expression cassette A comprising a promoter and an open reading frame coding for a controllable transcriptional modulator protein, at least one expression cassette B comprising a promoter and an open reading frame coding for a factor which regulates transcriptional activity of a baculovirus late and/or very late promoter, at least one expression cassette C comprising an open reading frame coding for said recombinant protein under the control of a baculovirus late and/or very late promoter responsive to the factor expressed by expression cassette B, and at least one transcription modulator response element in expression cassette C, wherein said controllable transcriptional modulator protein expressed by expression cassette A reversibly interacts with said transcriptional modulator response element(s) in said first condition, and interacts differently in said second condition, thereby modulating the transcription of expression cassette C.

In an alternative to the preceding embodiment, said baculovirus expression system comprises at least one expression cassette A comprising a promoter and an open reading frame coding for a controllable transcriptional modulator protein, at least one expression cassette B comprising a promoter and an open reading frame coding for a factor which regulates transcriptional activity of a baculovirus late and/or very late promoter, at least one expression cassette C comprising an open reading frame coding for said recombinant protein under the control of a baculovirus late and/or very late promoter responsive to the factor expressed by expression cassette B, and at least one transcription modulator response element in expression cassette B; wherein said controllable transcriptional modulator protein expressed by expression cassette A reversibly interacts with said transcriptional modulator response element(s) in said first condition, and interacts differently in said second condition, thereby modulating the transcription of expression cassette B. In a certain embodiment the baculovirus expression system further comprises an expression cassette B′ containing a promoter and an open reading frame coding for a said factor under the control of a weak promoter producing minimal levels of said factor still allowing baculovirus replication and amplification, or a modified factor leading to a minimal transcriptional activity of a baculovirus late and/or very late promoter, and wherein said modified factor still allows baculovirus replication and amplification. Although not mandatory, preferably said further expression cassette B′ is not inducible. In one embodiment said promoter in expression cassette C is a very late promoter and said factor encoded by expression cassette B is vlf-1 of AcMNPV (J Virol. 68, 7746-56) or a transcriptionally functional homolog thereof. Further said modified factor expressed by said optional expression cassette B′ is a modified vlf-1 protein, preferably a modified vlf-1 protein based on vlf-1 from AcMNPV (J Virol. 68, 7746-56) and is selected from the group consisting of C202Y mutant and vlf-1 comprising a cystein inserted between P23 and R24 (vcBsuSsevlf1).

Further embodiments and examples for expression cassettes A, B and C are found within the disclosure, definitions, claims and examples of co-pending application [PCT/EP2011/050996], such embodiments and examples being henceforth readily incorporated into the present invention by the person of ordinary skill following the disclosure herein. The disclosure of co-pending application [PCT/EP2011/050996], is hereby incorporated by referencing herein. Such co-pending application describes an inducible baculovirus expression system (“iBac”), which is suitable, with routine modification, to combine with the general method of the present invention. The iBac system overcomes the problem of high very late promoter driven (essentially constitutive) recombinant protein expression during scale up phase of baculovirus expression and the associated high selective pressure against the expression cassette encoding the recombinant protein, that can result in elimination and/or mutation of this cassette and overall reduced recombinant protein production. Briefly, the iBac system is an inducible baculovirus expression system that allows to repress or switch-off recombinant protein expression during scale up phase and to induce or switch-on recombinant protein expression during recombinant protein production phase. The combination of the iBac system together with the general method of the present invention provides particular additive and/or synergistic advantages in the production of recombinant protein by baculovirus expression in insect cells. For example, and as described above, such a combination can provide that when said first condition is maintained (ie during step (b) of the method of the present invention) expression of said recombinant protein is suppressed in addition to allowing production of said baculovirus virions; and when said second condition is maintained (ie during step (c) of the method of the present invention) expression of said recombinant protein is allowed in addition to suppressing production of said baculovirus virion. Disclosed herein are examples of particular combinations of certain embodiments of the general method of the present invention that provide for suppression of baculovirus virion production during production of recombinant protein; with certain embodiments of the iBac technology that provide for suppression of recombinant protein production during production of baculovirus virion. Following the disclosure herein, the person of ordinary skill will readily appreciate how other embodiments of the general method of the present invention may be combined with other embodiments of the iBac technology.

In a first particular embodiment of the invention as described herein the method comprises using a baculovirus expression system comprising at least one expression cassette X comprising a promoter that controls expression of a controllable transcriptional repressor protein; at least one expression cassette Y comprising a promoter, a transcriptional repressor response element and a nucleotide sequence coding for an RNAi effector targeting said at least one gene product essential for baculovirus virion assembly; at least one expression cassette Z comprising a promoter and an open reading frame coding for said gene product essential for baculovirus virion assembly, at least one expression cassette A, comprising a promoter and an open reading frame coding for a controllable transcriptional repressor protein, at least one expression cassette B comprising a promoter and an open reading frame coding for a factor which regulates transcriptional activity of a baculovirus late and/or very late promoter, at least one expression cassette C comprising an open reading frame coding for said recombinant protein under the control of a baculovirus late and/or very late promoter responsive to the factor expressed by expression cassette B, and at least one transcriptional repressor response element in expression cassette C, wherein said first condition maintained during scale up phase is repressive, thereby repressing transcription of said RNAi effector and allowing expression of said gene product essential for baculovirus virion assembly, and repressing expression of said recombinant protein; and said second condition maintained during recombinant protein production phase is non-repressive, thereby inducing transcription of said RNAi effector and suppressing expression of said gene product essential for baculovirus virion assembly, and inducing expression of said recombinant protein. Methods according to this first particular embodiment are also referred to herein as methods according to iVBac1A+iBac.

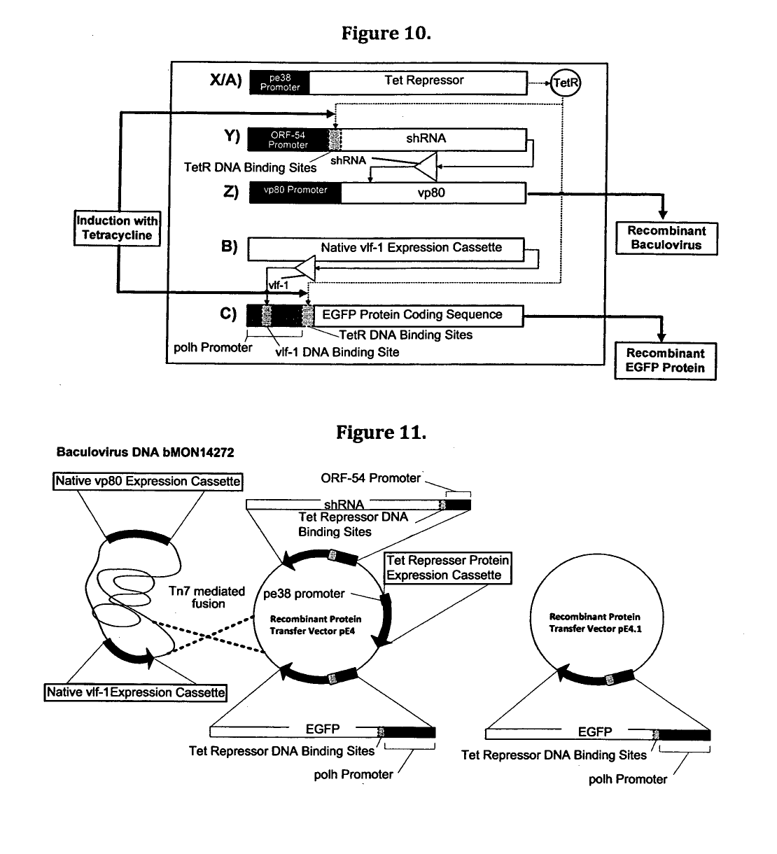

In such an embodiment, the at least one expression cassette X and the at least one expression cassette A may be the same or different. Preferably, the at least one expression cassette X and the at least one expression cassette A are coding for the same controllable transcriptional modulator protein and are either independent cassettes or one single expression cassette X/A. In the latter case said controllable transcriptional modulator protein expressed by expression cassette X/A reversibly interacts with said transcriptional modulator response elements in said first condition, and interacts differently in said second condition, thereby modulating the transcription of both expression cassette Y and expression cassette C. The term “a single expression cassette “X/A” as used herein refers to one expression cassette comprising a promoter and an open reading frame encoding the controllable transcriptional modulator protein (e.g. see FIG. 10), and hence can be considered as both expression cassette X and expression cassette A.

Although not strictly mandatory, in such an embodiment the promoter in expression cassette C may be a very late promoter and/or a preferred factor encoded by expression cassette B is very late factor vlf-1 of AcMNPV (J Virol. 68, 7746-56) or a transcriptionally functional homolog thereof, wherein the transcriptionally functional homolog of vlf-1 may be at least 70% identical on the amino acid level to vlf-1 of AcMNPV, more preferably it may be at least 75%, 80%, 85%, 90%, 95%, 98% or 99% identical on the amino acid level to vlf-1 of AcMNPV.

In a second (alternative) particular embodiment of the invention, the method comprises using a baculovirus expression system comprising at least one expression cassette X comprising a promoter that controls expression of a controllable transcriptional activator protein; at least one expression cassette Y comprising a minimal promoter, a transcriptional activator response element and a nucleotide sequence coding for an RNAi effector targeting said at least one gene product essential for baculovirus virion assembly; and at least one expression cassette Z comprising a promoter and an open reading frame coding for said gene product essential for baculovirus virion assembly, at least one expression cassette A, comprising a promoter and an open reading frame coding for a controllable transcriptional modulator protein, at least one expression cassette B comprising a promoter and an open reading frame coding for a factor which regulates transcriptional activity of a baculovirus late and/or very late promoter, at least one expression cassette C comprising an open reading frame coding for the recombinant protein under the control of a weak promoter responsive to the factor expressed by expression cassette B, and at least one transcription activator response element in expression cassette C, wherein said first condition maintained during scale up phase is non-activating, thereby repressing transcription of said RNAi effector and allowing expression of said gene product essential for baculovirus virion assembly, and repressing expression of the recombinant protein; and said second condition maintained during recombinant protein production phase is activating, thereby inducing transcription of said RNAi effector and suppressing expression of said gene product essential for baculovirus virion assembly, and inducing expression of the recombinant protein. The at least one expression cassette X and at least one expression cassette A may be the same or different. Preferably the at least one expression cassette X and at least one expression cassette A are coding for the same controllable transcriptional modulator protein and are either independent cassettes or one single cassette. In the latter case said controllable transcriptional modulator protein expressed by expression cassette X/A reversibly interacts with said transcriptional modulator response elements in said first condition, and interacts differently in said second condition, thereby modulating the transcription of both expression cassette Y and expression cassette C.

In one such embodiment, a preferred factor encoded by expression cassette B is very late factor vlf-1 of AcMNPV (J Virol. 68, 7746-56) or a transcriptionally functional homolog thereof, wherein the transcriptionally functional homolog of vlf-1 may be at least 70% identical on the amino acid level to vlf-1 of AcMNPV, more preferably it may be at least 75%, 80%, 85%, 90%, 95%, 98% or 99% identical on the amino acid level to vlf-1 of AcMNPV.

In a third (alternative) particular embodiment of the invention, the method comprises using a baculovirus expression system comprising at least one expression cassette X comprising a promoter and an open reading frame coding for a controllable transcriptional repressor protein, at least one expression cassette Z comprising a promoter and an open reading frame coding for a gene product essential for baculovirus virion assembly and at least one transcriptional repressor response element in expression cassette Z, wherein said controllable transcriptional repressor protein reversibly interacts with its transcriptional repressor response element(s) in said first condition, and interacts differently in said second condition, thereby directly or indirectly modulating the transcription of expression cassette Z, and further wherein said first condition maintained during scale up phase allows expression of said gene product essential for baculovirus virion assembly; and said second condition maintained during recombinant protein production phase suppresses expression of said gene product essential for baculovirus virion assembly. Methods according to this third particular embodiment are also referred to herein as methods according to iVBac2.

In certain of such embodiments the gene product essential for baculovirus virion assembly encoded by expression cassette Z is selected from the group consisting of vp80, vp39, vp1054 gp64, p74, p24 and p6.9. Preferably at least one gene product essential for baculovirus virion assembly is a vp80 baculovirus capsid protein. In a further embodiment the controllable transcriptional repressor protein is selected from the group consisting of TetR, CymR, trpR, MetJ, lac repressor protein and tox repressor protein. Preferably the controllable transcriptional repressor protein is TetR.

Preferably in such an embodiment, the promoter in expression cassette X is an early promoter, including early baculoviral promoters, such as pe38 promoter (Seq ID No: 2), or cellular promoters. The promoter in expression cassette Z preferably has a later time of onset of gene expression compared to the promoter in expression cassette X. Preferably the promoter in expression cassette Z is the native promoter of said gene product essential in baculovirus virion assembly, such as the native vp80 promoter (Seq ID No: 9) if said gene product essential in baculovirus virion assembly is vp80.

In a fourth particular embodiment of this aspect of the present invention, the baculovirus expression system comprises at least one expression cassette X comprising a promoter and an open reading frame coding for a controllable transcriptional repressor protein; at least one expression cassette X′ comprising a promoter and an open reading frame coding for a second transcriptional repressor protein, different to the controllable transcriptional repressor protein encoded by expression cassette X; at least one expression cassette Z comprising a promoter and an open reading frame coding for a gene product essential for baculovirus virion assembly, at least one expression cassette B comprising a promoter and an open reading frame coding for a factor which regulates transcriptional activity of a baculovirus late and/or very late promoter, at least one expression cassette C comprising an open reading frame coding for the recombinant protein under the control of a baculovirus late and/or very late promoter responsive to the factor expressed by expression cassette B, at least one transcriptional repressor response element in expression cassette X′ and expression cassette C responsive to said controllable transcriptional repressor protein encoded by expression cassette X, and at least one second transcriptional repressor response element in expression cassette Z responsive to said second transcriptional repressor protein encoded by expression cassette X′, wherein said controllable transcriptional repressor protein encoded by expression cassette X reversibly interacts with said transcriptional repressor response element in expression cassette X′ and C in said first condition, and interacts differently in said second conditions, thereby modulating the transcription of the second transcriptional repressor protein encoded by expression cassette X′ and of the recombinant protein encoded by expression cassette C, and further wherein said first condition maintained during scale up phase represses expression of said second transcriptional repressor protein encoded by expression cassette X′, thereby allowing expression of said gene product essential for baculovirus virion assembly, and represses expression of said recombinant protein; and said second condition maintained during recombinant protein production phase induces expression of said second transcriptional repressor protein encoded by expression cassette X′, thereby suppressing expression of said gene product essential for baculovirus virion assembly, and induces expression of said recombinant protein. Methods according to this fourth particular embodiment are also referred to herein as methods according to iVBac2+iBac.

In certain of such embodiments said baculovirus late and/or very late promoter in expression cassette C is a baculovirus very late promoter and/or said factor encoded by expression cassette B is vlf-1 of AcMNPV (J Virol. 68, 7746-56) or a transcriptionally functional homolog thereof under the control of its native promoter (Seq ID No: 9). In a further certain embodiment the second transcriptional repressor protein encoded by expression cassette X′ is a controllable transcriptional repressor protein or is a constitutive transcriptional repressor protein. A controllable transcriptional repressor protein expressed by expression cassette X′ may be one selected from the group consisting of TetR, CymR, trpR, MetJ, lac repressor protein and tox repressor protein, but must be different to the controllable transcriptional repressor protein encoded by expression cassette X and must bind to a different (second) transcriptional repressor response element as the controllable transcriptional repressor protein expressed by expression cassette X. In a preferred embodiment the second transcriptional repressor protein is the lac repressor protein.

Preferably in such an embodiment, the promoter in expression cassette X is an early promoter, including early baculoviral promoter such as pe38 promoter (Seq ID No: 2) or a cellular promoter. The promoter in expression cassette X′ preferably has a later time of onset of gene expression compared to the promoter in expression cassette X, such as the late baculovirus promoter ORF-54 (Seq ID No: 7). Preferably the promoter in expression cassette Z has a simultaneous or later time of onset of gene expression compared to the promoter in expression cassette X′, more preferably it is the native promoter of said gene product essential in baculovirus virion assembly, such as the native vp80 promoter (Seq ID No: 9) if said gene product essential in baculovirus virion assembly is vp80.

According to the invention, any one of said expression cassettes is contained in a transfer vector suitable for recombination with genomic or modified baculovirus DNA or is contained in a genomic modified baculovirus DNA.

In any of the embodiments of the method of the present invention said first condition may be maintained until the number of insect cells is between about 10⁸ and 10¹³ and/or until the number of baculovirus particles is between 10⁸-10¹³. Alternatively or in addition, said first condition is maintained until the total volume of culture of said insect cells is between 0.1 L and 10,000 L, and/or is maintained for a period of time that is between 1 day and 3 weeks.

In certain embodiment of the invention suppression of said expression of at least one gene product essential for baculovirus virion assembly does not substantially affect late and/or very late recombinant protein expression from said baculovirus expression system in comparison to late and/or very late expression from the baculovirus expression system without suppression of said expression of the at least one gene product essential for baculovirus virion assembly.

According to certain embodiments of the present invention, the method described herein comprises that the harvested recombinant protein contains suppressed or reduced, such as significantly or substantially suppressed or reduced, or contains essentially no (detectable) baculovirus virions compared to a method without suppressing expression of said at least one gene product essential for baculovirus virion assembly during recombinant protein production phase, wherein suppressed or reduced means 0-80% of the amount of baculovirus virions compared to the amount from a method without suppressing expression of said at least one gene product essential for baculovirus virion assembly during recombinant protein production phase, more specifically 0, 1, 5, 10, 15, 20, 25, 30, 35, 40, 50, 60, 70 or 80% of the amount of baculovirus virions compared to the amount from a method without suppressing expression of said at least one gene product essential for baculovirus virion assembly during recombinant protein production phase.

Optionally the method of the present invention comprises separation and/or purification of said harvested recombinant protein from contaminating components. The recombinant protein according to the invention may be a protein intended for use as a biopharmaceutical product such as a therapeutic or prophylactic agent, and/or may be a vaccine such as virus like particles from HPV or Influenza virus.

The insect cells comprising a baculovirus expression system as used in the method of the present invention can be obtained and/or provided by transfection or infection of the insect cells with the baculovirus expression system, such as prior to any other step of such method. The insect cells used in the method of the present invention are preferably selected from the group consisting of insect cells derived from Spodoptera frugiperda, Trichoplusia ni, Plutella sylostella, Manduca sextra and Mamestra brassicae; preferably the insect cell is selected from the group consisting of SF9, SF21, High Five™ Cells (BTI-TN-5B1-4) and Mimic™ Sf9 insect cells.

Typically, but not essentially, the inducible baculovirus expression system of the invention is based on the sequence derived from a nuclear polyhedosis virus (NPV), such as is based on the sequence of Autographa californica nuclear polyhedrosis virus (AcMNPV) (Virology 202 (2), 586-605 (1994), whose sequence can currently be retrieved under NCBI Accession No.: NC_{—}001623).

In another aspect the invention relates to a baculovirus expression system as disclosed, defined or claimed herein. Preferably, said baculovirus expression system is for use in (such as useful for or especially adapted for use in) the method of the present invention.

In a further aspect, the present invention relates to a baculovirus transfer vector comprising expression cassette Y of the baculovirus expression system as described, defined or claimed herein. Optionally, such transfer vector may further comprise expression cassettes X and/or C as described, defined or claimed herein.

In yet other aspects, the invention relates to a composite baculovirus DNA comprising the baculovirus expression system as described, defined or claimed herein and to an insect cell comprising a baculovirus expression system or a composite baculovirus DNA of the present invention.

A further aspect encompassed by the present invention is a kit, such as a kit for suppressing production of baculovirus virions during recombinant protein production phase in insect cells, wherein said kit comprises: (i) at least one expression cassette C⁽⁻⁾ containing a baculovirus late and/or very late promoter, wherein said expression cassette is intended for expressing a recombinant protein under the control of said promoter; (ii) at least one expression cassette Y as described, defined or claimed herein; and (iii) expression cassette X and/or A as described, defined or claimed herein. In some embodiments at least one of the expression cassettes is on a transfer vector, such as embodiments where the expression cassette are on different transfer vectors and in other embodiments the expression cassettes are on one transfer vector. In certain embodiments the expression cassette C⁽⁻⁾ further comprises a transcriptional modulator response element. In another embodiment expression cassette C⁽⁻⁾ further contains an open reading frame for expressing said recombinant protein under the control of said promoter.

As used herein “baculovirus expression system” refers to a system using baculoviruses coding for a recombinant protein allowing production of said recombinant protein in insect cells, particularly insect cell lines. A baculovirus expression system generally comprises all elements necessary to achieve recombinant protein expression in insect cells. The baculovirus expression system of the present invention further comprises the inducible expression control system as described herein. Thus, such a system may comprise a transfer vector and a modified baculovirus DNA, wherein the transfer vector can be fused to the modified baculovirus DNA to form a composite baculovirus DNA. The composite baculovirus DNA generally comprises all components of the baculovirus expression system. The baculovirus expression system generally does not contain a helper plasmid or a modified insect cell. Most of the presently used baculovirus expression systems are based on the sequence of Autographa californica nuclear polyhedrosis virus (AcMNPV) ((Virology 202 (2), 586-605 (1994), NCBI Accession No.: NC_{—}001623).

The term “recombinant protein” as used herein refers to any protein not naturally expressed under the control of a baculovirus late and/or very late promoter. In view of the inducible nature of the baculovirus expression systems provided herein, in certain embodiments the recombinant protein may also be a “toxic” protein for the host cell. Further the DNA sequence encoding the recombinant protein can be a naturally existing DNA sequence or a non-natural DNA sequence. The recombinant protein can be modified in any way. Non-limiting examples for modifications can be insertion or deletion of post-translational modification sites, insertion or deletion of targeting signals, fusion to tags, proteins or protein fragments facilitating purification or detection, mutations affecting changes in stability or changes in solubility or any other modification known in the art. In certain embodiments of the invention the recombinant protein is a biopharmaceutical product, which can be any protein suitable for therapeutic or prophylactic purposes in mammals. In some embodiments of the invention, the recombinant protein is a virus-like partial (VLP), including a VLP for use as a vaccine in mammals, including humans, such as for vaccination against influenza or HPV.

The term “scale up phase” as used herein refers to a phase of exponential increase in the number of recombinant baculoviruses from the starting point of small numbers of recombinant viruses, such as from a small pool on the order of millions of virus particles, to a large pool of viruses capable of driving large scale baculovirus expression. This requires intact virus replication and amplification as well as production of infectious virions to spread the infection. Scale up is typically carried out by allowing the recombinant viruses to replicate in sequentially larger insect cell numbers and/or culture volumes. Typically, the scale up phase is maintained until the number of insect cells is between about 10⁸ and 10¹³ of insect cells and/or until the number of baculovirus particles is between 10⁸-10¹³. The scale up phase may also be maintained until the total volume of culture is between 0.1 L and 10,000 L, and/or is maintained for a period of time that is between 1 day and 3 weeks. In scale up phase recombinant protein may be produced. However, in certain embodiments recombinant protein production can be suppressed during scale up phase.

The term “virus amplification” as used herein refers to the propagation of virus, such as serially passaging a virus to scale up number of virus particles starting from a clonal virus. Such propagation is typically conducted using insect cells.

The term “culturing” as used herein refers to maintaining insect cells in a suitable medium and under conditions allowing the cell to be maintained. This also includes up-scaling of virus producing insect cell cultures, comprising serial steps of harvesting virus and reinfecting larger insect cell cultures, or comprising a single step of infecting a cell culture at low M.O.I and allowing the multiple generations of baculoviral passage to occur in situ by maintaining the culture in log phase cell densities. In certain embodiments culturing comprises the generation of 10, 100, 1000 or even more liters of insect cell culture comprising the inducible baculovirus expression system of the invention. This also includes protein production phase, wherein culture conditions are optimized for recombinant protein production. This may comprise culturing cells in the absence of serum.

The term “recombinant protein production phase” as used herein typically follows the scale up phase when the desired culture volume, number of insect cells or number of baculoviral particles has been reached. As used herein it can be initiated by changing from a first condition to a second condition wherein the change in condition can be achieved by addition or deprival of a chemical inducer molecule or corepressor, or by a change of environmental factors such as light or temperature. Maintaining said cells under said second condition allows recombinant protein production and simultaneously suppresses or reduces expression of a gene product essential for baculovirus virion assembly, thereby reducing baculovirus virion production, by using a controllable transcriptional modulator either directly regulating expression of said gene product essential for baculovirion assembly or regulating transcription of an RNAi effector targeting the mRNA of said gene product essential for baculovirus virion assembly. Alternatively the controllable transcriptional modulator can further indirectly regulate expression of said gene product essential for baculovirion assembly, by directly regulating expression of a second transcriptional modulator protein, which again regulates expression of said gene product essential for baculovirus virion assembly. Optionally, maintaining said cells under said second condition further induces recombinant protein production by switching-on or increasing recombinant protein expression. Ideally, culture conditions with regard to cell density and culture medium conditions should be optimized for recombinant protein expression during recombinant protein production phase.

The term “baculovirus virions” as used herein refers to infectious and non-infectious baculovirus virions, including budded virions (BVs) and occlusion-derived virions (ODVs) (see Baculovirus Expression Vectors, ‘Reilly et al. Oxford University Press, New York, 1994).

The term “insect cell” typically relates to a cell line derived from Spodoptera frugiperda, Trichoplusia ni, Plutella sylostella, Manduca sextra and Mamestra brassicae. Preferably the insect cell is a cell from a standard or conventional insect cell line known in the art (e.g., Methods in Molecular Biology, Vol. 388. Murhammer, David W. (Ed.). 2nd ed., 2007). A standard or conventional insect cell line as used herein is a cell line commonly used for baculovirus expression systems that has not been further genetically engineered and does not comprise additional recombinant DNA other than the composite baculovirus DNA. Preferably, the insect cell is selected from the group consisting of SF9, SF21, High Five™ Cells (BTI-TN-5B1-4) and Mimic™ Sf9 insect cells. A particular suitable example is the insect cell Sf9, wherein Sf9 is a clonal isolate of IPLB-SF21AE.

The term “at least one gene product essential for baculovirus virion assembly” as used herein refers to the protein encoded by a gene, which upon inactivation or deletion of the gene results in a baculovirus phenotype with suppressed or reduced numbers of baculovirus virions, including budded virions (BVs) and occlusion-derived virions (ODVs). These may include capsid proteins or proteins required at any step of capsid assembly or for transport of the virions out of the cell. Such a gene may be identified by deletion of said gene and analyzing the baculovirus phenotype as known in the art. Additionally RNAi technology as described herein may be used to silence expression of said gene and analyzing the baculovirus phenotype as known in the art. For example, the methods of Marek et al (Biotechnology and Bioengineering 108, 1056-1067), involve infecting insect cells with a baculovirus, transfecting the same cells with a dsRNA identical in sequence to a gene suspected of being essential for baculovirus virion assembly, and looking for the formation of BVs and ODVs using plaque assays, PCR and electron microscopy. Alternatively, the method as described in Example 4 can be used to screen for optimal shRNA effector molecules and targets.

Ideally the suppression of expression of the at least one gene product essential for baculovirus virion assembly does not or not significantly affect very late gene expression from said baculovirus in comparison to very late gene expression from the respective wild type baculovirus expression system. Preferably the gene essential for baculovirus virion assembly is selected from the group consisting of vp80, vp39, vp1054, gp64, p74, p24 and p6.9, even more preferably the gene product essential for baculovirus virion assembly is the vp80 baculovirus capsid protein and optionally one further gene product essential for baculovirus virion assembly.

“Inducible expression control system” as used in the present invention refers to a system wherein the expression and/or transcription of a protein or RNAi effector of interest can be reversibly switched-on or increased in a first condition and switched-off or decreased in a second condition, thereby directly or indirectly suppressing or allowing protein expression. The system allows expression of said at least one gene product essential for baculovirus virion assembly under one condition and suppresses expression of at least one gene product essential for baculovirus virion assembly under a second condition during production of the recombinant protein of interest. Optionally, it further suppresses expression of said recombinant protein under one condition and allows expression of the recombinant protein under a second condition.

The term “inducible” as used herein generally refers to the expression and/or transcription of any protein or RNAi effector of interest that is switched-on or increased, which is directly mediated by a (controllable) transcriptional modulator protein, reversibly interacting with its transcriptional modulator response element(s) in a first condition, and interacting differently in a second condition. In case the (controllable) transcriptional modulator protein is a (controllable) transcriptional repressor protein it is not binding to its transcriptional repressor response element regulating said protein or RNAi effector expression in an induced state. In case the (controllable) transcriptional modulator protein is a controllable transcriptional activator protein it is binding to its transcriptional activator response element regulating said protein or RNAi effector expression in an induced state. Preferably, this induction is reversible by changing conditions, thereby repressing protein or RNAi effector expression.

The term “repressed” as used herein generally refers to the expression and/or transcription of any protein or RNAi effector of interest that is switched-off or decreased, which is directly mediated by a (controllable) transcriptional modulator protein, reversibly interacting with its transcriptional modulator response element(s) in a first condition, and interacting differently in a second condition. In case the (controllable) transcriptional modulator protein is a (controllable) transcriptional repressor protein, it is binding to its transcriptional repressor response element regulating said protein or RNAi effector expression in a repressed state. In case the (controllable) transcriptional modulator protein is a controllable transcriptional activator protein, it is not binding to its transcriptional activator response element regulating said protein or RNAi effector expression in a repressed state. Preferably, this induction is reversible by changing conditions, thereby inducing protein or RNAi effector expression.

The term “suppressing” as used herein refers to any interference of protein expression or baculovirus virion production, resulting in reduced expression levels of said protein or reduced amounts of baculovirus virions compared to the respective wild-type baculovirus expression systems. This includes repressed protein expression directly controlled by a controllable transcriptional modulator protein or indirectly controlled by a controllable transcriptional modulator via a second transcriptional modulator. It also includes the presence of effective amounts of RNAi effector specifically targeting mRNA encoding said protein, wherein effective amounts means that expression levels of said protein are substantially reduced, i.e., by at least 70, 75, 80, 85, 90, 95 or 100% compared to the respective wild-type baculovirus expression system.

The term “allowing” as used herein refers to the expression of any protein that is not interfered with. This includes wild type expression and expression cassettes and induced protein expression directly controlled by a controllable transcriptional modulator protein or indirectly controlled by a controllable transcriptional modulator via a second transcriptional modulator. This further includes the presence of no or non-effective amounts of RNAi effector specifically targeting said protein, wherein non-effective amounts means that expression levels of said protein are not substantially decreased, i.e., are decreased by less than 20, 10, 5, 2.5, 1, 0.5 or 0% compared to the respective wild-type baculovirus expression systems.

For the purposes of the present invention, “(responds/interacts) differently”, with respect to controllable the transcriptional modulator protein, includes the meaning of where said modulator protein maintains a particular occupancy on the transcriptional modulator response element in one condition, and a lower occupancy on said response element in a second condition. For example, in one condition the binding constant of said modulator protein changes such that its effective occupancy on said response element is lower, and hence said modulator protein becomes (far) more likely to dissociate from, or not to associate with, said response element, including to such an extent that said modulator protein can be considered to no longer (effectively) interact or bind to said response element, and in particular to no longer (effectively) modulate the transcription of expression cassette Y (iVBac1A/1B) and/or expression cassettes C (iVBac1A/1B+iBac), or alternatively no longer (effectively) repressing the transcription of expression cassette Z, comprising a repressor response element (iVBac2), or expression cassette X′ (iVBac2+iBac).

As used herein the term “expression cassette” refers to an entity made up of a gene and the sequences controlling its expression. An expression cassette comprises at least a promoter sequence, an open reading frame encoding for a protein, and a 3′ untranslated region. The expression cassette can be part of a vector DNA, plasmid DNA, bacmid DNA, genomic DNA, a virus, a cell or any other DNA/RNA or DNA/RNA containing organism.

The term “promoter” as used herein is a region of DNA that facilitates the transcription of a particular gene. Promoters are typically located adjacent to the genes they regulate, on the same strand and upstream (towards the 5′ region of the sense strand). Preferably, the promoters referred to herein are baculovirus promoters. Also preferably the promoter in expression cassette X and/or expression cassette A as described herein has an earlier time of onset of gene expression than the promoter in any of the other expression cassettes described herein. Wherein the term “time of onset of gene expression” as used herein is to be understood relative to the baculovirus infection in the sense of a postinfection time of onset of gene expression.

The term “open reading frame” (ORF) as used herein is a portion of an organism's genome which contains a DNA sequence that could potentially encode a protein. In a gene, ORFs are located between the start-code sequence (initiation codon) and the stop-code sequence (termination codon).

The expression cassette X of the present invention comprises a promoter and an open reading frame coding for a controllable transcriptional modulator protein.

A “controllable transcriptional modulator protein” as used herein can be a controllable transcriptional activator or a controllable transcriptional repressor, activating or repressing transcription, respectively, upon reversibly interacting with the respective transcriptional modulator response element. The reversible interaction, e.g., reversible binding, can be controlled by the presence of an inducer molecule or by a change in environmental conditions. The DNA bound transcriptional activator typically activates transcription through interaction with the basal transcription machinery or with other factors that upregulate transcription. Typically the DNA bound transcriptional repressor sterically inhibits transcription initiation or transcription elongation.

The expression cassette Y of the present invention contains a promoter, a transcriptional modulator response element and a nucleotide sequence coding for an RNAi effector targeting said at least one gene product essential for baculovirus virion assembly.

The term “transcriptional modulator response element” as used herein is a consensus DNA binding site for a controllable transcriptional modulator, typically, but not necessarily located in or adjacent to promoters. The transcriptional modulator response element is a transcriptional activator response element in case of a controllable transcriptional activator and a transcriptional repressor response element in the case of a controllable transcriptional repressor. Typically one controllable transcriptional modulator interacts with the transcriptional modulator response elements of one expression cassette, i.e., expression cassette Y, thereby regulating transcription of said expression cassette, i.e., expression cassette Y. However, in the present invention one controllable transcription modulator can also interact with several transcription modulator response elements in one and also in different expression cassettes. One non-limiting example would be a controllable transcription modulator protein interacting with the modulator response element in expression cassette Y and in expression cassette C, thereby regulating transcription of both expression cassettes Y and C. An alternative non-limiting example would be a controllable transcription modulator protein interacting with the modulator response element in expression cassette X′ and in expression cassette Z.

Preferably, in the present invention the transcriptional modulator response element is located in an expression cassette, activating or repressing transcription of the same expression cassette. In principle, the transcriptional modulator response element can be located anywhere within the expression cassette, such as e.g., in the 5′ untranslated region, in the promoter region, between the promoter and the transcription initiation site, within the open reading frame or in the 3′ untranslated region of the expression cassette. It is also possible that the transcriptional modulator response element reversibly interacting with the controllable transcriptional modulator is located outside that same expression cassette, as long as said interaction activates transcription of said expression cassette. A non-limiting example would thus be a transcriptional repressor response element within a polh enhancer-like element to repress polh driven transcription from expression cassette C. Another non-limiting example would be a tetR DNA binding site (Seq ID No: 10) just downstream of the late baculovirus promoter ORF-54 (Seq ID No: 7).

In certain embodiments of the present invention, the transcriptional modulator response element is comprised in or in proximity to (adjacent to) the promoter of said expression cassette in expression cassette Y. In other embodiments of the present invention, the transcriptional modulator response element is in expression cassette Y and expression cassette C. For the purposes of the present invention, a transcriptional modulator response element is considered “in proximity” (or adjacent) to a promoter if it is situated downstream or upstream of said promoter at a distance of between 10 and 1,000 bp, and preferably less than 100 bp, from the transcription start-site of said promoter.

In particular embodiments of the present invention, the transcriptional modulator response element is a transcriptional repressor response element in expression cassette Y or in expression cassette Y and expression cassette C, preferably in or in proximity to (adjacent to) the promoter of expression cassette Y and/or expression cassette C.

In another particular embodiments of the present invention, the transcriptional modulator response element is a transcriptional repressor response element in expression cassette Z or in expression cassette X′ and expression cassette C and a second transcriptional repressor response element responsive to the second transcriptional repressor protein expressed by expression cassette X′ in expression cassette Z, preferably in or in proximity to (adjacent to) the promoter of expression cassette X′, expression cassette Z and/or expression cassette C.

In a further particular embodiment of the present invention, the transcriptional modulator response element is a transcriptional activator response element in expression cassette Y or in expression cassette C, preferably in or in proximity to (adjacent to) the promoter of expression cassette Y and/or expression cassette C. To prevent transcription of expression cassette Y in the absence of the transcriptional activator protein the promoter is a minimal promoter. To prevent transcription of expression cassette C in the absence of the transcriptional activator protein the promoter is a weak promoter.

As used herein the term “minimal promoter” refers to any promoter resulting in no or minimal transcription without additional activation. A non-limiting example for a minimal promoter is the ie2 minimal promoter of from (Protein Expression and Purification 42, 236-245).

The term “RNAi effector” encoded by expression cassette Y of the present invention relates to a RNA interference effector, which is a double stranded RNA molecule leading to post-transcriptional depletion of the mRNA transcript of expression cassette Z; preferably one or more RNA molecules(s) specifically targeting at least one gene product essential for baculovirus virion assembly encoded by expression cassette Z. Said RNAi effector can be a short interfering RNA (siRNA), a short hairpin RNA (shRNA), a long hairpin RNA (IhRNA) or a polycystronic shRNA. Preferably the RNAi effector is a shRNA or a polycystronic shRNA, even more preferably a shRNA. In certain embodiments one RNAi effector targets one gene product essential for baculovirus virion assembly, wherein the RNAi effector can form multiple siRNAs specifically targeting different target sequences of the same open reading frame coding for a gene product essential for baculovirus virion assembly. In another embodiment one RNAi effector targets two or more gene products essential for baculovirus virion assembly, e.g., two or three gene products essential for baculovirus virion assembly.

In certain embodiments of the invention one, two or three gene product(s) essential for baculovirus virion assembly are targeted by an RNAi effector, preferably two gene products essential for baculovirus virion assembly, thereby suppressing baculovirus virion production even more. One way this could be achieved is using one RNAi effector encoded by one expression cassette Y, specifically targeting several, i.e., two or three (or more) different gene product(s) essential for baculovirus virion assembly, wherein the RNAi effector can be either a IhRNA or polycistronic shRNA targeting multiple separate mRNAs or genes. Alternatively this could also be achieved using two or three (or more) different RNAi effector(s) encoded by two or three (or more) separate expression cassettes Y each and with each of the separate RNAi effector(s) specifically targeting a different gene product essential for baculovirus virion assembly.

The expression cassette Z of the present invention contains a promoter and an open reading frame coding for said gene product essential for baculovirus virion assembly. In certain embodiments the at least one gene product essential for baculovirus virion assembly is selected from the group consisting of vp80, vp39, vp1054, gp64, p74, p24 and p6.9. Preferably at least one gene product essential for baculovirus virion assembly is vp80 baculovirus capsid protein (Seq ID No: 8) and even more preferably under the control of its native late promoter (Seq ID No: 9). According to the invention, there can be one, two or more expression cassettes Z, preferably two or three expression cassettes Z, wherein the open reading frame of one expression cassette Z codes for one gene product essential for baculovirus virion assembly and the open reading frame of the second and/or third expression cassette Z codes for another gene product essential for baculovirus virion assembly.

According to the present invention, expression of the at least one gene product essential in baculovirus virion assembly expressed by expression cassette(s) Z can be directly or indirectly repressed (suppressed), and in certain embodiments during recombinant protein production phase, expression levels of said gene product essential in baculovirus virion assembly encoded by expression cassette Z is lower as compared to a respective wild-type baculovirus expression system. Preferably, according to the present invention, during recombinant protein production phase protein expression levels of the respective gene product essential in baculovirus virion assembly encoded by expression cassette Z should be less than about 30% of wild type levels, more preferably less than 20% or even less than 10% of wild-type levels. In fact, in some systems, even larger suppression factors may be achieved, particularly if the timing of the repressor or activator protein has an earlier time of gene onset compared to the expression from cassettes Y (iVBac1A and iVBac1B) or expression cassette Z (iVBac2). Preferably more than one gene product essential for protein production are directly or indirectly repressed (suppressed).

The term “early promoter” as used herein is a promoter used for baculovirus late gene expression, for example to express genes between about 1 and 6 hours post infection. The term “late promoter” as used herein is a promoter used for baculovirus late gene expression, for example to express genes between about 6 and 24 hours post infection, such as between about 12 and 18 hours post infection. The term “very late promoter” as used herein is a promoter used for baculovirus very late gene expression, for example to express genes between about 18 and 72 hours post infection, such as between about 18 and 24 or about 24 and 72 hours post infection. Two highly expressed very late genes have been characterized, the polyhedrin and the p10 gene, and their respective very late promoters have been named polh and p10 promoter. Examples for AcMNPV-derived sequences serving as very late promoters are given as Seq ID No: 15 (polh) and Seq ID No: 20 (p10). During the very late phase of infection both genes undergo a burst of transcription, leading to accumulation of their respective RNAs and proteins in the cell (Virology 248, 131-138). Very late promoters are thought to differ from late promoters primarily by the presence of a burst sequence, a sequence downstream of the transcription start site that is 90% A and T (J. Virol. 79, 1958-1960).

As will be appreciated by the person of ordinary skill, the term “baculovirus late and/or very late promoter” comprises any promoter that functions as a late and/or functions as a very late promoter for a baculovirus, and includes such promoters that comprise nucleic sequences derived from a late, or from a very late, promoter that is present in the genome of a wild-type baculovirus. In this context, “derived from” includes nucleic acids sequences that are greater than 80%, such as 85%, 90%, 95%, 99% or 100% identical over about 5, 10, 15, 20, between 20 and 30 or between 30 and about 50 bp. Accordingly, synthetic promoters, that are not native to a given baculovirus species—such as hybrid promoters that contain fragments of both the late Pcap promoter (Gene. 91 (1990) 87-94) and a very late promoter such as polh or p10. Such hybrid promoters may be activated by baculovirus factors (such as those encoded by expression cassette B) at both late and very late stages of infection.

According to certain embodiments of the invention the timing of the promoter in expression cassettes X, Y and Z can be timely staggered. Preferably the promoter in expression cassette X has an earlier time of onset of gene expression than the promoter in expression cassette Y, and the promoter in expression cassette Y has an earlier or simultaneous time of onset of gene expression than the promoter in expression cassette Z. Even more preferably the promoter in expression cassette Z is the native promoter of said gene product essential for baculovirus virion assembly. The expression “earlier time of onset” is relative to baculovirus infection relating to an earlier postinfection time of onset. Although not strictly mandatory, promoters of expression cassettes according to the invention are preferably baculoviral promoters. A detailed analysis regarding postinfection time of onset has been described by Jiang et al., (Journal of Virology, 2006, p 8989-8999) and can be used as a guidance. Preferably, the promoter in expression cassette X is a baculovirus early promoter.

Non-limiting examples for a baculovirus early promoters are the pe38 promoter (Seq ID No: 2), the me53 promoter, the lef3 promoters and the he65 promoter. Typically baculovirus early promoter comprise at least one early or CATG motif. Also typically baculovirus early promoter use cellular transcription factors. Hence also cellular promoter can be used in expression cassette X that are transcribed in insect cells and would be considered as early promoters. Preferably the promoters in expression cassettes Y and expression cassette Z are baculovirus late and/or very late promoters with a similar time of onset. More preferably the baculovirus late and/or very late promoters in expression cassette Y has an earlier onset compared to the baculovirus late and/or very late promoters in expression cassette Z. Even more preferably the promoter in expression cassette Z is the native promoter of said gene product essential for baculovirus virion assembly.

According to another embodiment of the invention the timing of the promoter in expression cassettes X and Z or X, X′ and Z can also be timely staggered. Preferably the promoter in expression cassette X has an earlier time of onset of gene expression than the promoter in expression cassette Z and the promoter of the optional expression cassette X′ has a later time of onset of gene expression than the promoter of expression cassette Y and a simultaneous, but preferably earlier time of onset of gene expression than the promoter of expression cassette Z. In this embodiment the promoter in expression cassette Z preferably is the native promoter of said gene product essential for baculovirus virion assembly. Although not strictly mandatory, promoters of expression cassettes according to the invention are preferably baculoviral promoters. A detailed analysis regarding postinfection time of onset has been described by Jiang et al., (Journal of Virology, 2006, p 8989-8999) and can be used as a guidance. Preferably, the promoter in expression cassette X is a baculovirus early promoter and the promoters in expression cassettes Z and/or expression cassette X′ are baculovirus late and/or very late promoters.

In accordance with one embodiment of the present invention the baculovirus expression system as used in the method comprises at least one expression cassette X, expression cassette Y and one expression cassette Z and a transcriptional modulator response element in expression cassette Y, reversibly interacting with a controllable transcriptional modulator protein in one condition, and (reacting/interacting) differently in a second condition, thereby modulating transcription of the RNAi encoded by expression cassette Y, which interferes with expression of the protein encoded by expression cassette Z.

Alternatively, the baculovirus expression system as used in another embodiment of the method comprises at least one expression cassette X and at least one expression cassette Z and a transcriptional modulator response (repressor) element in expression cassette Z, reversibly interacting with a controllable transcriptional modulator protein in one condition, and (reacting/interacting) differently in a second condition, thereby modulating expression of the at least one gene essential for baculovirus virion assembly encoded by expression cassette Z.

As mentioned herein, expression of expression cassette Z can be directly or indirectly regulated by a controllable transcriptional modulator interacting with its respective modulator response element. Under said second conditions said gene product essential for baculovirus virion assembly under the control of a weak promoter encoded by expression cassette Z is produced at lower than wild-type levels of said gene product essential for baculovirus virion assembly, thereby suppressing baculovirus virion production. Preferably two or three gene products essential for baculovirus virion assembly are produced at lower than wild-type levels of said two or three gene products essential for baculovirus virion assembly, thereby even more suppressing baculovirus virion production.

The expression cassette A of the present invention comprises a promoter and an open reading frame coding for a controllable transcriptional modulator protein. The promoter and/or the open reading frame coding for a controllable transcriptional modulator protein may the same or different as in expression cassette X. In a preferred embodiment the expression cassette A is the same as the expression cassette X. Even more preferably the expression cassette A is identical with expression cassette X, forming one single expression cassette X/A. Further embodiments of expression cassette A can be found in co-pending application [PCT/EP2011/050996].

The expression cassette B when used in the present invention contains a promoter and an open reading frame coding for a factor which regulates transcriptional activity of a baculovirus late and/or very late promoter, and in certain embodiments wherein transcriptional activity of the baculovirus late/and or very late promoter decreases with lower than wild-type levels of said factor in insect cells.

In other certain embodiments of the present invention, the expression cassette B is native to and/or is found in the genome of a wild-type baculovirus.

In certain embodiments of the present invention, said factor that regulates transcriptional activity of the baculovirus (late and/or very late) promoter stimulates, can stimulate and/or is capable of stimulating transcriptional activity of said promoter.

In particular embodiments of the present invention, said factor is one which regulates transcriptional activity of a baculovirus very late promoter (referred to herein as “VLTF”), such as where transcriptional activity of the baculovirus very late promoter decreases with lower than wild-type levels of said VLTF in insect cells.

The factor (eg VLTF) can be a naturally occurring factor (such as VLTF)—ie it can be considered a native factor such as one expressed by the expression cassette B that is native or naturally occurring to the baculovirus system—or the factor may be a transcriptionally functional homolog thereof.

According to some embodiments of the present invention, expression from expression cassette B can be directly regulated by a controllable transcriptional modulator interacting with a transcriptional modulator response element, wherein the transcriptional modulator response element is preferably inside the expression cassette B. In the latter case the native factor of the baculovirus expression system is, in certain embodiments, replaced by a factor expressed by expression cassette B. In other embodiments, a native transcription factor may be modified or its transcription may be modified, such as described herein, so that its transcription regulatory activity is reduced.

In alternative embodiments of the present invention, the factor encoded by expression cassette B and the baculovirus late and/or very late promoter (in expression cassette C or Z) that it regulates come from a different baculovirus species or strain to the baculovirus that form the other parts of the inducible baculovirus expression system, such that any native transcription factors expressed by the other parts of the inducible baculovirus expression system do not regulate the transcriptional activity of said promoter, and its transcriptional activity is (predominately) modulated by the factor encoded by expression cassette B, and (predominately) not by any native transcription factors expressed by the other parts of the inducible baculovirus expression system.

The term “a factor which regulates transcriptional activity of a baculovirus late and/or very late promoter”, (such as, when referring to a very late promoter, abbreviated “VLTF” herein), refers to any molecule, such as a peptide, polypeptide or protein, capable of regulating transcriptional activity of a baculovirus late and/or very late promoter, and in certain embodiments (including such as when the controllable transcriptional modulator is a controllable transcriptional activator) wherein its transcriptional activity decreases with lower than wild-type levels of the factor in insect cells. One example for a suitable factor according to the present invention is vlf-1.

In preferred embodiments of the invention, the factor is vlf-1 of AcMNPV (J Virol. 68, 7746-56) or a transcriptionally functional homolog thereof. Vlf-1 interacts with the burst sequence of p10 and polyhedrin regulatory regions and selectively up-regulates genes under the control of the very late promoters.

Other baculovirus proteins that may act as a factor (like a VLTF) include FP25, ie-1, Lef-2, Lef-4, PK-1, Ac43, polh enhancer-like binding proteins, or any functional homologs of the above proteins; and in certain embodiments (including such as when the controllable transcriptional modulator is a controllable transcriptional activator), provided that lower levels of such a factor (like a VLTF) lead to lower than wild-type levels of recombinant protein expression from expression cassette C in insect cells while still allowing virus replication.

Yet other baculovirus proteins that may act as a suitable factor in the present invention (in this case that regulate transcription of a baculovirus late promoter) include Lef-1, Lef-2, Lef-3, Lef-4, Lef-5, Lef-6, Lef-7, Lef-8, Lef-9, Lef-10, Lef-11, Lef-12, IE-1, IE-2, Ac69, Ac38, Ac36, p47, p143, p35, DNAPOL, HCF-1. Further information on such factors can be found in J. Virology, 27: 10197-10206. It is known by the person of ordinary skill that some factors (such as Lef-2 and Lef-4) regulate baculovirus late and also regulate very late promoters.

Methods for determining if a factor regulates transcriptional activity of a baculovirus late and/or very late promoter have been described, such as exemplarily for vlf-1 by Yang and Miller (Virology 248, 131-138). Briefly, the gene of interest is mutated, deleted or, if the gene is essential for replication, cloned under the control of a weak promoter. The weak promoter can be any weak promoter described in the literature for the respective factor or can be generated as described for vlf-1 accordingly, for example by inserting a DNA fragment containing the hsp70 promoter between the factor open reading frame and its original promoter in opposite orientation to the original promoter. In case the gene of interest is essential for the replication of baculovirus, the viability of the virus indicates that sufficient amounts of the gene are produced by the recombinant virus. Amounts of the potential factor and polyhedrin-driven (or another late and/or very late promoter) expression can be easily monitored in cells infected with baculovirus with the gene of interest mutated or deleted or under the control of a weak promoter and compared to cells infected with “wild type” baculovirus at the same M.O.I. by, e.g., immunoblot examination. The person skilled in the art will appreciate that any other method for detecting quantitatively or semi-quantitatively the product of the gene of interest and the late and/or very late gene product (for example polyhedrin or alternatively p10) can be applied. Detection can be at the transcriptional or translational level, detecting mRNA or protein levels, respectively. Non-limiting examples for detection methods are flow cytometry, microscopy, real-time PCR, immuno- or Western blotting, ELISA and Northern blotting. Further, the polyhedrin or, alternatively, p10 gene or any suitable late and/or very late baculovirus gene, can be replaced by a reporter gene such as a genes encoding chloramphenicol acetyltransferase (CAT), a fluorescent protein like GFP, YFP or their enhanced analogues, a luminescent protein like luciferase or any other protein that is easily detectable. Methods for detecting and quantifying reporter gene expression are well known in the art. Further embodiments of expression cassette B and suitable factors, may be found in co-pending application [PCT/EP2011/050996].

The term “wild type virus” as used herein refers to the phenotype of the typical form of a species as it occurs in nature including expression systems derived therefrom. In the case of AcMNPV the wild type virus is encoded by the sequence of NCBI accession number NC_{—}001623 (Virology 202 (2), 586-605 (1994)) and wild type expression systems are based on this sequence.

As used herein a “wild type expression system” is not inducible (and/or repressible), meaning that expression of a gene product essential for baculovirus virion assembly is not controlled by an engineered system of transcriptional repressor or activator proteins and their respective response elements, neither directly nor indirectly via one or more RNAi effector(s) or a second transcriptional repressor. Additionally the promoter in expression cassette Z is the native (original) promoter of the respective gene product essential in baculovirus virion assembly. Described in terms of expression cassettes as defined herein a wild type expression system does not comprise an expression cassette X (X/A) and/or expression cassette Y (iVBac1A and iVBac1B) or does not comprise an expression cassette X (X/A) (iVBac2) and or expression cassette X′. Additionally, in combination with the iBac system the expression of the recombinant protein under the control of a late and/or very late promoter is not controlled by an engineered system of transcriptional repressor or activator proteins and their respective response elements, neither directly nor indirectly via any factors such as baculovirus (or non-baculovirus) transcription factors acting on late and/or very late promoters, particularly a factor such as vlf-1. Additionally the factor (such as VLTF) is full-length and not modified in a way that interferes with its transcriptional activity, potential virus replication activity or stability of mRNA or protein and is expressed under the control of its original promoter. Described in terms of expression cassettes as defined herein a wild type expression system does not contain an expression cassette A and/or its corresponding transcriptional modulator response element as defined herein, but contains an expression cassette B with an open reading frame coding for a factor (such as VLTF) under the control of its native promoter and an expression cassette C as defined herein, wherein the factor (such as VLTF) of expression cassette B is full-length and not modified in a way that interferes with its transcriptional activity, potential virus replication activity or stability of mRNA or protein, and the respective promoter is the original promoter or a functional homolog thereof. A wild type expression system further comprises an expression cassette Z, wherein said gene product essential for baculovirus virion assembly is under the control of its native promoter. Further the gene product essential for baculovirus virion assembly encoded by expression cassette Z is full-length and not modified in a way that interferes with its transcriptional activity, virion assembly or stability of mRNA or protein.

The abbreviation M.O.I as used herein refers to the multiplicity of infection and is the ratio of infectious virus particles to infection targets (e.g. cells). For example, when referring to a group of cells inoculated with infectious virus particles, the multiplicity of infection or M.O.I is the ratio defined by the number of infectious virus particles deposited in a well divided by the number of target cells present in that well.

The term “transcriptionally functional homolog” as used herein relates to a protein factor (such as VLTF) that does not have the same amino acid sequence than the protein factor (such as VLTF) it refers to, but is functionally identical or similar in its transcriptional activation of a baculovirus late and/or very late promoter. This means that the protein factor (such as VLTF) can be replaced with its transcriptionally functional homolog in a recombinant baculovirus expression system without any substantial changes in expression levels of the recombinant protein under the control of the late and/or very late promoter. No substantial changes in this context means that the transcriptional activity of a very late promoter in the presence of the transcriptionally functional homolog should be at least 80% of transcriptional activity of the wild type factor (such as VLTF), preferably 85%, 90%, 95%, 100% or even more than 100% the transcriptional activity of the wild type factor (such as VLTF). This includes fragments of the full-length protein factor (such as VLTF) with a transcription activity from a very late promoter similar to wild type full-length protein factor (such as VLTF).

The expression cassette C of the present invention contains an open reading frame coding for a recombinant protein under the control of a baculovirus late and/or very late promoter. In certain embodiments expression cassette C further comprises a transcriptional modulator response element functionally interacting with the expression product of expression cassette A which, in certain embodiments, is a transcriptional repressor response element.

According to the present invention, expression of the recombinant protein expressed by expression cassette C can be directly or indirectly repressed, and in certain embodiments wherein, in the repressed (off) state, expression levels of said recombinant protein of (encoded by) expression cassette C is lower as compared to a non-inducible baculovirus expression system with an expression cassette B containing an open reading frame coding for said factor (such as VLTF) under the control of its original promoter, and an expression cassette C as defined above, but without an expression cassette A and/or its corresponding transcriptional modulator response element. Preferably, according to the present invention, transcription from late and/or very late promoters in expression cassette C in the repressed (off) state should be less than about 50% of wild type levels, more preferably less than 30%, 20% or even less than 10% of wild type levels. In fact, in some systems, even larger repression factors may be achieved, particularly if the timing of the repressor or activator protein expression is synchronized with the expression from cassettes C.

According to the invention the recombinant protein is under the control of a baculoviral late and/or very late promoter, preferably under the control of a very late promoter, even more preferably under the control of the polyhedrin Seq ID No: 15 or p10 promoter Seq ID No: 20. As mentioned earlier herein, expression of expression cassette C can be directly regulated by a controllable transcriptional modulator interacting with its respective modulator response element. In certain embodiments the transcriptional modulator protein is a transcriptional activator protein and expression cassette C contains an open reading frame encoding a recombinant protein under the control of a weak promoter and a transcriptional activator response element. Further embodiments for expression cassette C may be found in co-pending application [PCT/EP2011/050996].

As used herein the term “weak promoter” refers to any promoter producing low recombinant protein expression. An example for a weak promoter is derived from the vhspRVvlf1 construct described in (Virology 248, 131-138). The weak promoter derived from vhspRVvlf1 was created by inserting a fragment from the Drosophila melonogaster HSP70 heat shock protein promoter 3 bps upstream of the vlf-1 translation start codon in opposite direction to the vlf-1 promoter. This method can be used accordingly for any factor to generate a weak promoter from its original promoter. Any non-native factor promoter sequence that results in lower transcription of said factor than wild-type levels can likewise be used as a weak promoter in accordance with the present invention. Preferably, the weak promoter produces less than about 75%, and more preferably less than 50%, 40%, or even 30% of the protein expression compared expression under its original promoter.

According to the present invention the expression cassettes described herein can be contained in a transfer vector suitable for fusion, i.e., recombination with genomic or modified baculovirus DNA or is contained in a genomic modified baculovirus DNA.

In a preferred embodiment the transcriptional modulator protein is a transcriptional repressor protein and expression cassette C contains an open reading frame encoding a recombinant protein under the control of a very late promoter and a transcriptional repressor response element.

The term “providing insect cells comprising a baculovirus expression system” as used herein refers to insect cells that have been treated according to any method known to the person skilled in the art to bring the DNA encoding the baculovirus expression system or the baculovirus comprising the DNA encoding the baculovirus expression system into an insect cell. This comprises methods such as transfection, microinjection, transduction and infection. Methods for transfecting DNA into insect cells are known to the person skilled in the art and can be carried out, e.g., using calcium phosphate or dextran, by electroporation, nucleofection or by lipofection.

In the baculovirus expression systems of the present invention recombinant genes are cloned into the baculovirus genome either through recombination of modified baculovirus DNA and recombinant gene-containing plasmids in insect or bacteria cells, or through in vitro generation. Using any of these methods, sufficient recombinant protein-encoding viruses or DNA are generated to infect or transfect, respectively, insect cell culture volumes in the order of a few milliliters.

The insect cell according to the present invention, including an intermediate host insect cell, can be any insect cell supporting baculovirus production. Examples for insect cells supporting baculovirus production are cells derived from Spodoptera frugiperda, Trichoplusia ni, Plutella xylostella, Manduca sexta, and Mamestra brassicae. Preferred insect cells in the context of the invention are the IPLB-SF21AE cell or its clonal isolate, the Sf9 cell.

Typically, baculovirus expression systems are derived from nuclear polyhedrosis viruses (NPV). While in principle all baculovirus expression systems can be modified to work in the context of the present invention, a preferred inducible baculovirus expression system is based on Autographa californica nuclear polyhedrosis virus (AcMNPV). Examples of other preferred viruses include any of the multiple nucleocapsids per envelope (MNPV) subgenera of the NPV genera of the Eubaculovirinae subfamily (occluded Baculoviruses) of the Baculoviridae family of insect viruses.

There are a number of commercial systems for expressing recombinant proteins using baculovirus, all based on AcMNPV. They include flashBAC™ (Oxford Expression Technologies EP1144666), BackPack™ (BD Biosciences Clontech), BacVector® 1000/2000/3000 (Novagen®), BAC-TO-BAC® (Invitrogen™ U.S. Pat. No. 5,348,886), and BaculoDirect™ (Invitrogen™). All these baculovirus-based insect cell expression systems are essentially based on expressing recombinant proteins by placing them under the control of very late baculovirus promoters, namely the polh and/or p10 promoters. Any of these baculovirus expression systems could be conveniently modified to comply with the present invention by incorporating controllable transcriptional activators or repressors into the system as described herein, thereby directly or indirectly controlling recombinant protein expression. Accordingly, the baculovirus expression systems of the present invention can be based on any one of the commercially or academically available baculovirus expression systems.

There are a vast number of well studied controllable transcriptional activators and repressors and their respective response elements available in the literature that can be used according to the invention. They come from prokaryotes, eukaryotes, or were created through molecular biological techniques. The majority of commercially available inducible systems have been engineered to improve or combine natural systems to suit the needs of a wide range of specialized scientists. For example, DNA binding domains from prokaryotes have been combined with activation domains from eukaryotes. Similarly, a wide array of synthetic chemical inducers or co-repressors have been described which function better than their natural structural analogues.

A controllable transcriptional modulator can be an inducer molecule controlled transcriptional activator protein, an inducer molecule (corepressor) controlled transcriptional repressor protein, a physically controlled transcriptional activator protein or a physically controlled transcriptional repressor protein.

Suitable non-limiting examples for inducer molecule-controlled transcriptional activators and their inducers are the protein metallothionein (MT) binding DNA sequences called metal responsive element (MREs) in the presence of its inducer, metals such as copper (CuSO₄, Nucl. Acids Res. 16, 1043-1061); AMT1, another metal responsive transcriptional activator (Proc. Natl. Acad. Sci. 88, 6112-6116); steroid-inducible transcriptional activators, glucocorticoid receptor (GC) proteins, binding GREs in the presence of steroid inducers such as cortisol or its structural analogue dexamethasone (Proc. Natl. Acad. Sci. 90, 5603-5607); estrogen receptor (ER) binding its DNA response elements under the influence of a wide range of molecules (Pharmacol. Rev. 58, 773-781); the alcohol-dependent transcription activator AlcR (Mol. Microbiol. 20, 475-488), the chimeric protein tTA and rtTA of the Tet-off and Tet-on system, respectively, binding to tetracycline response elements (TREs) controlled by tetracycline or derivatives such as doxycycline (Annu. Rev. Genet. 36, 153-73, PNAS 89, 5547-5551); the CAP protein and its inducer cAMP (JMB 293, 199-213); the AP-1 proteins and its inducer phorbol esters (Mol. Pharm. 56, 162-169); and WRKY1, WRKY2, and WRKY3 activating transcription in the presence of the oligopeptide elicitor Pep25 (EMBO J. 15, 5690-700).

Suitable but non-limiting examples for inducer molecule controlled transcriptional repressors and their inducers (corepressors) are the tetracyclin repressor protein TetR and its corepressor tetracycline or derivatives thereof such as doxycycline (EMBO 3, 539-43); the CymR repressor protein and its corepressor p-cumate (J. Bacteriol. 179, 3171-3180); the trpR repressor and its corepressor tryptophan (PNAS 79, 3120-3124); the PurR repressor and its corepressor guanine or hypoxanthine (Cell 83, 147-155), the MetJ repressor protein and its corepressor SAM (Proc. Natl. Acad. Sci. 106, 5065-5069); the lac repressor protein and its corepressor lactose and structural analogues thereof such as IPTG (C. R. Biol. 328, 521-48); and the tox repressor protein and its corepressor transition metals (PNAS 92, 6803-7). In particular embodiments of the invention, the presence of an inducer molecule stimulates expression of the recombinant protein that is under the control of the baculovirus late and/or very late promoter in expression cassette Y and/or expression cassette C.

Transcription can also be activated or repressed by environmental factors such as light or temperature. Non-limiting examples for physically-induced transcriptional activator proteins and their inducers are heat shock transcription factor (HSF) binding to heat shock promoter elements (HSE) with consensus nGAAnnTTCn, heat shock proteins (HSPs) such as the temperature responsive HSP system in Drosophila (Nature 327, 727-730; Nature 368, 342-344) with HSP70 and associated proteins such as Hap46 (Proc. Natl. Acad. Sci. 96, 10194-10199) or Drosophila HSP70 homologs such as Ssa 1 (Mol. Microbiology 62, 1090-1101). Other species have comparable systems such as the HSF1 transcriptional activator from Arabidopsis (Biol. Chem., 384, 959-963). Photocaged derivatives of hydroxytamoxifen and guanidine tamoxifen have been synthesized that selectively antagonize estrogen receptor (ER) activated transcription at classic estrogen response elements (ChemBioChem 5, 788-796).

Non-limiting examples for physically induced transcriptional repressor proteins are the bacterial hrcA repressor protein reversibly binding its CIRCE DNA element in response to changes in temperature (J. Bacteriol. 182, 14-22). Further, RheA is a temperature sensitive protein from Streptomyces albus (PNAS 97, 3538-3543) and HSF-4a is human temperature sensitive repressor protein (J. Cell. Biochem. 82, 692-703).

Preferably the controllable transcriptional repressor protein of the invention is selected from the group consisting of TetR, CymR, trpR, MetJ, lac repressor protein and tox repressor protein. The controllable transcriptional activator protein is preferably selected from the group consisting of metallothionein (MT), AMT1, Glucocorticoid receptor protein (GC), Estrogen receptor, AlcR, tetR-VP16, tTA, CAP, AP-1, WRKY1, WRKY2 and WRKY3. More preferably the controllable transcriptional activator protein is a modified ecdysone receptor protein.

In other embodiments of the invention, the transcriptional modulator protein, is not a hormone receptor, for example is one derived from a bacterial/prokaryotic DNA binding protein, such as one that is, or is derived from, a bacterial/prokaryotic transcriptional modulator protein.

In other embodiments of the present invention, the transcriptional repressor response element is not a hormone receptor response element, and in certain such embodiments the transcriptional modulator response element is derived from a bacterial/prokaryotic transcriptional modulator response element”.

As mentioned above, in preferred embodiments of the invention the factor which regulates transcriptional activity of a baculovirus very late and/or late promoter is a factor which regulates transcriptional activity of a baculovirus very late promoter. More preferably said factor is one known as “very late factor 1” (vlf-1) or a transcriptionally functional homolog thereof. The term a “transcriptionally functional homolog” thereof refers to a protein that shows a comparable transcriptional activity as vlf-1 of AcMNPV (J Virol. 68, 7746-56) of wherein comparable means at least 80%, preferably 85%, 90%, 95%, 100% or >100% of the transcriptional activity of “wild type” vlf-1. Methods for determining transcriptional activity of a protein are known in the art and can be determined for example by methods described herein. Even more preferably the transcriptionally functional homolog of vlf-1 further is at least 70% identical to vlf-1 of AcMNPV on the amino acid level, more preferably at least 75%, 80%, 85%, 90%, 95%, 98% or 99% identical to vlf-1 on the amino acid level. According to the invention transcriptionally functional homologs of vlf-1 also include fragments of vlf-1 protein with the transcriptional activity being at least 80%, preferably 85%, 90%, 95%, 100% or >100% of the transcriptional activity of vlf-1 of AcMNPV determined as described above.

In certain embodiments the vlf-1 gene is expressed under the control of a promoter, wherein promoter means “original” or a functional homolog thereof. The “original” promoter of vlf-1 from AcMNPV is given in (J Virol. 68, 7746-56) and is one preferred embodiment of the present invention.

The method according to the invention allows production of recombinant protein in the presence of suppressed or reduced levels of baculovirus virions. The method of the invention comprises harvesting recombinant protein comprising less than 40, 30, 20, 10, 5, 1, 0.5 or 0.1% of baculovirus virions compared with recombinant protein expressed in a wild-type baculovirus expression system.

In another aspect, the present invention relates to a recombinant nucleic acid, such as one comprising expression cassette Y(−) as defined or otherwise described herein. For example, such a recombinant nucleic acid can comprise at least: (i) the promoter of expression cassette Y of the inducible baculovirus expression system of the present invention; (ii) a transcriptional modulator response element; and (iii) a cloning site, such as one for inserting, an RNAi effector. Suitable cloning/insertion sites for the recombinant nucleic acids of the invention will be readily known to the person of ordinary skill and include (multiple) cloning sites that can be digested by one or more restriction enzyme, and/or recombination-based insertion sites such as those used for the “Gateway” cloning system of Invitrogen of that use Cre-Lox system.

In certain embodiments of the recombinant nucleic acids of the invention, the transcription of a nucleic acid inserted into the cloning site is under the control of the promoter therein, and such transcription may be modulated by a controllable transcriptional modulator protein reversibly interacting, in one condition, with the transcriptional modulator response element of said recombinant nucleic acid. In other embodiments, said transcriptional modulator response element is in, or in proximity (adjacent) to, said promoter.

In particular embodiments of the recombinant nucleic acids of the invention, said transcriptional modulator response element is not a hormone receptor response element. In other embodiments, said transcriptional modulator response element binds to a bacterial/prokaryotic controllable transcriptional modulator protein and/or is (or is derived from) a bacterial/prokaryotic transcriptional modulator response element.

In a particular embodiment of the recombinant nucleic acids of the invention, said transcriptional modulator response element may bind to, may be capable of binding, be controlled by or may be controlled by a controllable transcriptional repressor protein.

In other certain embodiments, the recombinant nucleic acids of the invention may further comprise a nucleotide sequence coding for an RNAi effector.

In another aspect, the present invention relates to a transfer vector, such as a bacmid, comprising a recombinant nucleic acid of the invention. In certain embodiments, the vector of the invention is useful for fusion with baculovirus DNA, including with modified baculovirus DNA, and in particular such embodiments the vector is a baculovirus transfer vector. As will be appreciated by the person of ordinary skill, such vector will also comprise other features that assist the maintenance and/or replication of the vector in a cell, such as in a cell described herein.

In other certain embodiments, the transfer vector further comprises expression cassettes X or A and/or expression cassette C(−), into which an open reading frame encoding a recombinant protein can be cloned and wherein the expression of said recombinant protein is under the control of a baculovirus late and/or very late promoter. For example, such a recombinant nucleic acid can comprise at least: (i) the promoter of expression cassette C of the inducible baculovirus expression system of the present invention; (ii) a transcriptional modulator response element; and (iii) a cloning site, such as one for inserting an open reading frame coding for the desired recombinant protein. Suitable cloning/insertion sites for the recombinant nucleic acids of the invention will be readily known to the person of ordinary skill and include (multiple) cloning sites that can be digested by one or more restriction enzyme, and/or recombination-based insertion sites such as those used for the “Gateway” cloning system of Invitrogen of that use Cre-Lox system.

In yet another aspect, the present invention relates to a composition that includes a recombinant nucleic acid of the invention. A composition of the present invention includes any mixture of two or more components one of which includes a recombinant nucleic acid of the invention as defined, claimed or otherwise described herein. In certain embodiments of such aspect, the composition is a two component mixture including such nucleic acid and at least one other component useful for the construction, and/or practice of the methods using, the inducible baculovirus expression system of the invention. Such other component may include a nucleic acid encoding expression cassettes X or A and/or expression cassette C(−). Alternatively, such other component(s) may comprise the controllable transcriptional modulator protein, such as the controllable transcriptional repressor protein that reversible interacts with the transcriptional modulator response element of said nucleic acid. In other embodiments of this aspect of the invention, the composition may be a complex mixture, such as that of, or otherwise found in, a cell-free transcription/translation system, a cell-extract or an intact cellular environment. In particular such embodiments, the inventive composition that includes a recombinant nucleic acid of the invention is a cell, such as a bacterial, yeast, insect or mammalian cell, for example such a cell comprised in in-vitro or industrial tissue culture or storage.

In a further aspect, the present invention relates to composite baculovirus DNA that comprises expression cassette Y as described herein. Optionally said composite baculovirus DNA further comprises any of the expression cassettes described herein.

In particular embodiments, the recombinant nucleic acid, the vector, the composition or the composite baculovirus DNA of the invention, further comprises expression cassette X of the inducible baculovirus expression system of the invention.

In yet a further aspect, the present invention relates to a method of suppressing production of baculovirus virion in an insect cell, said method comprising: a) providing insect cells of the invention; and b) maintaining said insect cells under conditions wherein the expression of at least one gene product essential for baculovirus virion assembly encoded by expression cassette Z of said inducible baculovirus expression system is suppressed by inducing transcription of an RNAi effector encoded by expression cassette Y of said baculovirus expression system.

In certain embodiments of any of the various methods of the present invention, said repression is brought about by the controllable transcriptional modulator protein (encoded by expression cassette X and/or A of said baculovirus expression system) reversibly interacting with the transcriptional modulator response element (of said baculovirus expression system) in one condition, and (reacting/interacting) differently in a second condition, thereby modulating the transcription of expression cassette Y and/or expression cassette C of said inducible baculovirus expression system. In particular such embodiments, said repression is, is effected by, or is otherwise directly or indirectly caused by, a transcriptional repressor protein binding to a transcriptional repressor response element in expression cassette Y and/or expression cassette C.

In other particular embodiments of the methods of the invention, interaction of the controllable transcriptional modulator protein to its transcriptional modulator response element causes (or effects) said repression, preferably wherein said transcriptional modulator response element is in expression cassette Y of said inducible baculovirus expression system and optionally additionally in expression cassette C.

In other certain embodiments of the methods the controllable transcriptional modulator protein is one that can be controlled by an inducer molecule, and said repression comprises the presence of, or in alternative embodiments the absence of, said inducer molecule.

In particular embodiments of the methods of the invention that comprise the induction of expression of an RNAi effector or a recombinant protein, said induction comprises the presence of an inducer molecule with the inducible baculovirus expression system, preferably by addition of the inducer molecule to said culture or maintenance conditions.

As will now be apparent to the person of ordinary skill, the various methods of the present invention have particular advantages in the context of large scale (such as industrial) production of recombinant protein in the presence reduced contaminating baculovirus virions. Accordingly, in certain embodiments of the various methods of the invention, the method includes a step of culturing insect cells that comprises conditions under which infectious baculovirus is amplified in said insect cells, under which the number of insect cells increases and/or under which the number of baculovirus particles is increased. In those methods including a step comprising culture conditions for baculovirus amplification, said conditions comprise between 2 and about 10 rounds of virus amplification, such as comprising 3, 4, 5, 6 or 8 rounds of virus amplification.

In certain embodiments of the method claims, the culture conditions are maintained (and/or repeated), or the various steps of the method are repeated until the number of insect cells is between about 10⁸ and 10¹³, such as between about 10⁹ and 10¹² cells, and/or until the number of baculovirus particles is between 10⁸ and 10¹³, such as between about 10⁹ and 10¹² virus particles. In alternative embodiments, said conditions are maintained until the total volume of culture is between about 0.1 L and 10,000 L, such as between about 100 and 1,000 L, and/or are maintained for a period of time that is between about 1 day and 3 weeks, such as between about 3 days and 1 week after the introduction of the inducible baculovirus system of the invention into the insect cell or the provision (and/or start of culture) of the insect cell comprising such a system.

In particular embodiments of the various aspects of the present invention, expression cassette X′ further comprises a transcriptional repressor response element, preferable in or in proximity (adjacent) to the promoter of expression cassette X′ and an open reading frame coding for a (second) transcriptional repressor protein, wherein said transcriptional modulator response element reversible interacts with a controllable transcriptional modulator protein in one condition, and (reacts/interacts) differently in a second condition, thereby modulating the transcription of expression cassette X′, and said second transcriptional repressor protein encoded by expression cassette X′ modulates transcription of expression cassette Z, wherein expression cassette Z additionally comprises a (second) transcriptional repressor response element, responsive to said second transcriptional repressor protein. Without being bound by theory, the inclusion of a transcriptional modulator response element in expression cassette X′, and its use to control (over) expression of the second transcriptional modulator protein, is thought to further reduce expression of said gene essential for baculovirus virion assembly expressed by expression cassette Z, enabling to reduce levels of contaminating baculovirus virions during recombinant protein production phase even more effectively. In certain of such embodiments, the controllable transcriptional modulator protein is a controllable transcriptional repressor protein.

Other aspects of the present invention relate to a kit, such as a kit of parts, that includes a plurality of components for the construction and/or use of an inducible baculovirus expression system of the present invention. Such plurality of components may be presented, packaged or stored separately. For example, they may be isolated from one another by being held in separate containers. Accordingly, one embodiment of such a kit of the invention comprises at least two components that include (preferably separately): (i) the recombinant nucleic acid, vector, composition or the composite baculovirus DNA of the invention; and (ii) at least one other component for the construction and/or use of an inducible baculovirus expression system. Such components, although held separately, may be boxed or otherwise associated together to aid storage and/or transport, and such association may include additional components. In particular embodiments of such kits of the invention, at least one of the second (or additional) components may comprise: (i) the expression cassette X of the inducible baculovirus expression system of any of the claims above, or a vector or a cell that comprises said expression cassette; (ii) an insect cell, preferably one selected from the selected from the group consisting of insect cells derived from Spodoptera frugiperda, Trichoplusia ni, Plutella xylostella, Manduca sexta, and Mamestra brassicae, such as an insect cell that is a IPLB-SF21AE cell or its clonal isolate Sf9; (iii) an inducer molecule that modulates the reversible interaction of a controllable transcriptional modulator protein with a transcriptional modulator response element; and/or (iv) instructions describing how to construct and/or use the inducible baculovirus expression system of the present invention, and/or to practice any of the various methods of the present invention.

It will be appreciated that the following examples are intended to illustrate but not to limit the present invention. Various other examples and modifications of the foregoing description and examples will be apparent to a person skilled in the art after reading the disclosure without departing from the spirit and scope of the invention. In fact, those of ordinary skill in the art will appreciate that baculovirus systems and components thereof and methods other than those specifically described herein can readily be employed in the practice of this invention as broadly described herein without undue experimentation. All baculovirus systems and components thereof and methods that can be readily adapted to the practice of this invention or that are recognized in the art to be functionally equivalents of the specific baculovirus systems and components thereof and methods disclosed herein are intended to be encompassed by the appended claims.

Moreover, all references cited are incorporated by reference in their entirety to the extent that they are not inconsistent with the description herein.

### EXPERIMENTS

### Example 1

### iVBac1A

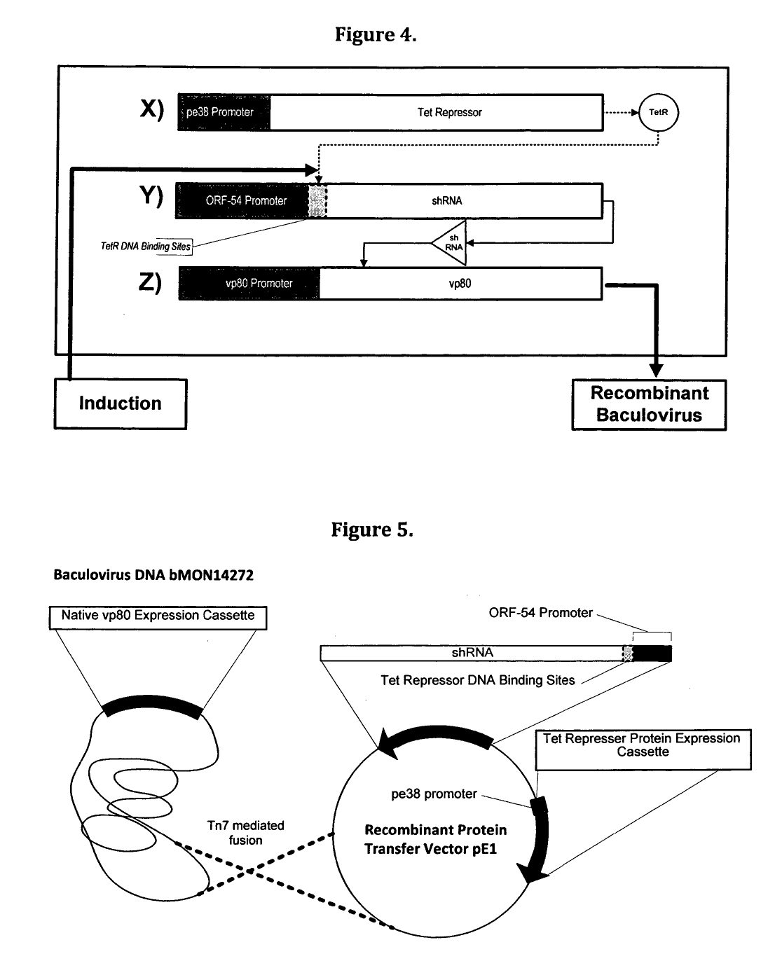

As shown in FIG. 4 an inducible transcriptional repressor protein, the tet repressor protein (tetR; Seq ID No: 1), is the product of expression cassette X. Expression cassette X is under control of the early baculovirus pe38 promoter (Seq ID No: 2). Expression cassette Y encodes an shRNA, targeting through RNA interference the RNA product of expression cassette Z. To target the baculovirus vp80 gene, the shRNA sequence is designed by use of the following algorithms: Gene Link shRNA Design, (GeneLine), BLOCK-iT™ RNAi Designer (Invitrogen), GenScript siRNA Construct Builder (GeneScript). Other algorithms may be analogously used. The shRNA sequence used in this example is given in Seq ID No: 3 and alternative shRNA sequences may include Seq ID No: 4, Seq ID No: 5, or Seq ID No: 6. Expression cassette Y is under control of the late baculovirus promoter ORF-54 (Seq ID No: 7). Expression cassette Z encodes the native baculovirus capsid protein vp80 (Seq ID No: 8) under control of its native late promoter (Seq ID No: 9). tetR has a DNA binding site (transcriptional repressor response element; Seq ID No: 10) just downstream of the ORF-54 promoter in expression cassette Y. In the absence of tetracycline, tetR remains bound to its DNA binding site in Y and thereby inhibits transcription of shRNA targeting vp80 RNA from expression cassette Z. When tetracycline is provided to the system, tetR releases from its DNA binding site and transcription of shRNA targeting vp80 RNA is induced. Baculovirus virion production by baculovirus-infected insect cells in the presence and absence of tetracycline is quantified by measuring virus titers through a virus plaque assay (J. Gen. Virol. 36, 361-364) and PCR (Biotechnology and Bioengineering 108, 1056-1067) or through an end point dilution assay (see Baculovirus Expression Vectors, O'Reilly et al. Oxford University Press, New York, 1994).

The strategy for creation of composite baculovirus containing the expression cassettes X, Y, and Z is described schematically in FIG. 5. Here Z, the native vp80 expression cassette, is located on the baculovirus DNA bMON14272 (J. Virology 67, 4566-4579). Cassettes X, coding for tetR and Y, coding for the shRNA from Seq ID No: 3, are located on a transfer vector called pE1 (FIG. 5). Transfer vector pE1 is based on pFastbac Dual (Invitrogen) and is constructed as follows. Briefly, the HindIII-KpnI fragment provided in Seq ID No: 11 is prepared by gene synthesis, and is subcloned into HindIII-KpnI digested pFastbac Dual.

Composite baculovirus DNA containing expression cassettes X, Y and Z is created by fusing transfer vector pE1 with bMON14272 (FIG. 5). This is carried out by transforming pE1 into DH10BAC (Invitrogen) E. coli cells harboring the bacmid bMON14272 and the helper plasmid pMON712417. Tn7 mediated recombination in the DH10BAC cells then mediates the fusion of bMON14272 with pE1 as described in (J. Virology 67, 4566-4579).

To create composite baculovirus bMON14272-pE1 and baculovirus bMON14272-pFastBAC Dual (negative control), composite bacmid DNA is isolated from DH10BAC following Tn7 transposition with either pFastbac Dual or pE1, respectively, and transfected into SF21 insect cells according to (Nature Methods 3, 1021-1032). bMON14272-pE1 and bMON14272-pFastbac Dual initial transfection virus is then harvested and used for subsequent baculovirus virion suppression tests.

Baculovirus virion suppression tests are carried out as follows. 10 milliliters of SF21 cells in suspension culture (Nature Methods 3, 1021-1032) at density 0.5×10⁶ cells/ml are infected at multiplicity of infection (M.O.I) of 1.0 with either bMON14272-pE1 or bMON14272-pFastbac Dual initial transfection virus either in the presence, or absence of 5 μg/ml tetracycline. Following 72 hours incubation, cells are centrifuged for 10 minutes at 4000 rpm in a tabletop centrifuge and virus supernatant is harvested. Infectious virus titers are then determined through plaque assay (J. Gen. Virol. 36, 361-364) or an end point dilution assay (see Baculovirus Expression Vectors, O'Reilly et al. Oxford University Press, New York, 1994). The production of infectious virus particles together with non-infectious virus particles and occluded virus particles is quantified by PCR (Biotechnology and Bioengineering 108, 1056-1067) carried out on both infected cells and virus supernatant.

Quantification of effectiveness of tetracycline-mediated suppression of generation of baculovirus virions through shRNA targeting of the essential virus capsid protein vp80 is carried out by comparing the results of the end point dilution assay or the plaque assay and PCR with and without tetracycline-mediated expression of shRNA. It is anticipated that tetracycline-mediated suppression of vp80 will significantly reduce the levels of Baculovirus virion production with bMON14272-pE1, while addition of tetracycline will have little or no effect on baculovirus virion production with bMON14272-pFastbac Dual.

### Example 2

### iVBac1B

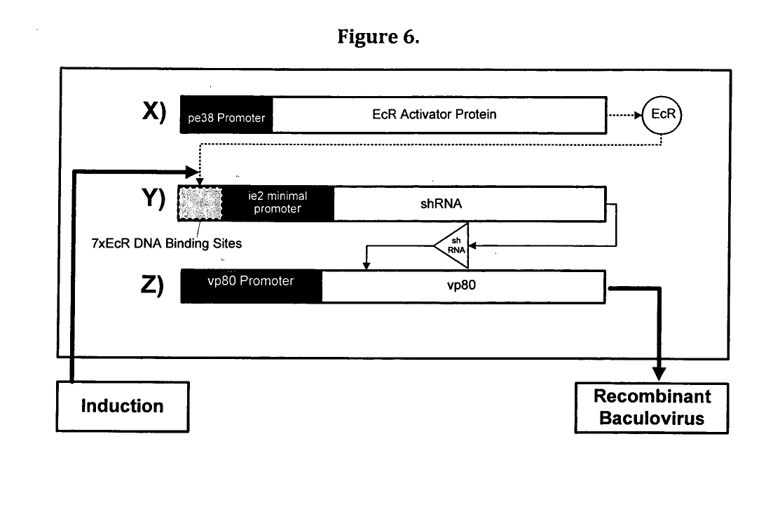

As shown in FIG. 6 an inducible transcriptional activator protein, a modified ecdysone receptor protein here called “EcR” (Protein Expression and Purification 42, page 238, GAL4-EcR:DEF), is the product of expression cassette X. Expression cassette X is under control of the early baculovirus pe38 promoter (Seq ID No: 2). Expression cassette Y encodes an shRNA targeting through RNA interference the RNA product of expression cassette Z. To target the baculovirus vp80 gene, the shRNA sequence is designed by use of the following algorithms: Gene Link shRNA Design, (GeneLine), BLOCK-iT™ RNAi Designer (Invitrogen), GenScript siRNA Construct Builder (GeneScript). Other algorithms may be analogously used. The shRNA sequence used in this example is given in Seq ID No: 3 and alternative shRNA sequences may include Seq ID No: 4, Seq ID No: 5, or Seq ID No: 6. Expression cassette Y is under control of the ie2 minimal promoter (Protein Expression and Purification 42, 236-245). Expression cassette Z encodes the native baculovirus capsid protein vp80 (Seq ID No: 8) under control of its native late promoter (Seq ID No: 9). EcR has DNA binding sites (Protein Expression and Purification 42, page 238, plasmid EcRe-CAT) just upstream of the ie2 minimal promoter in expression cassette Y. In the absence of the synthetic non-steroidal ecdysone agonist RG-102240, EcR remains unbound to its DNA binding site in Y and thereby does not stimulate transcription of shRNA targeting vp80 RNA from expression cassette Z. When RG-102240 is provided to the system, EcR binds to its DNA recognition sequence and transcription of shRNA targeting vp80 RNA is induced. Infectious virus titers are then determined through an end point dilution assay (see Baculovirus Expression Vectors, O'Reilly et al. Oxford University Press, New York, 1994). Alternatively, baculovirus virion production by baculovirus-infected insect cells in the presence and absence of RG-102240 may be quantified by measuring virus titers through a virus plaque assay (J. Gen. Virol. 36, 361-364) and PCR (Biotechnology and Bioengineering 108, 1056-1067).

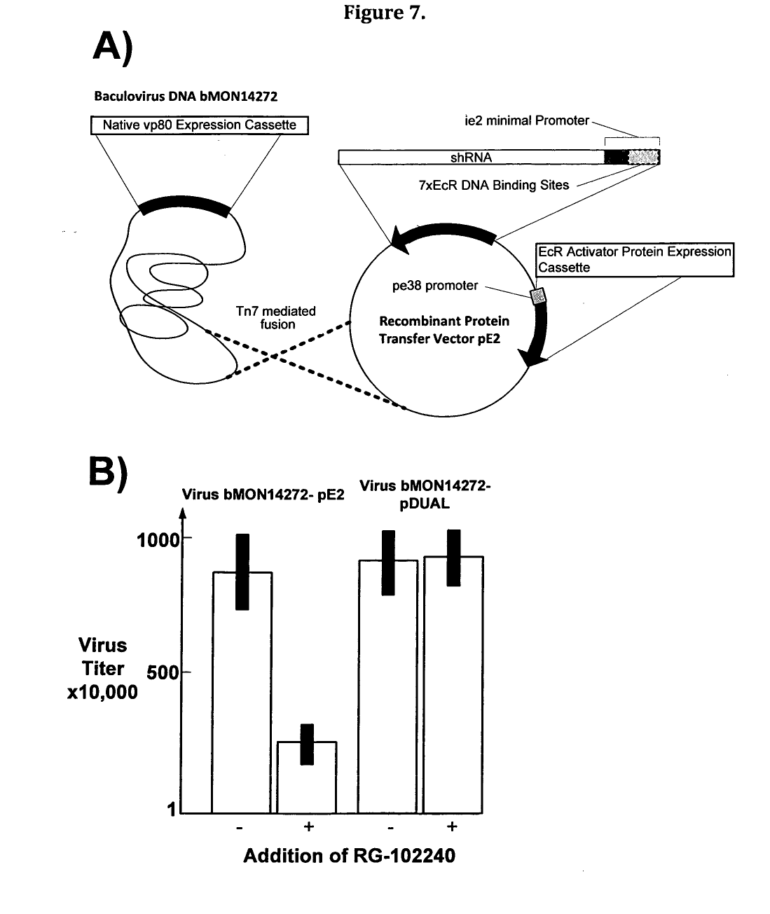

The strategy for creation of composite baculovirus containing of the expression cassettes X, Y, and Z is described schematically in FIG. 7A. Here Z, the native vp80 expression cassette, is located on the baculovirus DNA bMON14272 (J. Virology 67, 4566-4579). Cassettes X, coding for EcR, and Y, coding for the shRNA, are located on a transfer vector called pE2 (FIG. 7A). Transfer vector pE2 is based on pFastbac Dual (Invitrogen) and is constructed by gene synthesis by the same strategy as for pE1. Composite baculovirus DNA containing expression cassettes X, Y and Z is created by fusing transfer vector pE2 with bMON14272 (FIG. 7A). This is carried out by transforming pE2 into DH10BAC (Invitrogen) E. coli cells harboring the bacmid bMON14272 and the helper plasmid pMON712417. Tn7 mediated recombination in the DH10BAC cells then mediates the fusion of bMON14272 with pE2 as described in (J. Virology 67, 4566-4579).

To create composite baculovirus bMON14272-pE2 and baculovirus bMON14272-pFastBAC Dual (negative control), composite bacmid DNA is isolated from DH10BAC following Tn7 transposition with either pFastbac Dual or pE2, respectively, and transfected into SF21 insect cells according to (Nature Methods 3, 1021-1032). bMON14272-pE2 and bMON14272-pFastbac Dual initial transfection virus is then harvested and used for subsequent baculovirus virion suppression tests.

Baculovirus virion suppression tests are carried out as follows. 10 milliliters of SF21 cells in suspension culture (Nature Methods 3, 1021-1032) at density 0.5×10⁶ cells/ml are infected at multiplicity of infection (M.O.I) of 1.0 with either bMON14272-pE2 or bMON14272-pFastbac Dual initial transfection virus either in the presence, or absence of 0.5 μM RG-102240 (FIG. 7B). Following 72 hours incubation, cells are centrifuged for 10 minutes at 4000 rpm in a tabletop centrifuge and virus supernatant is harvested. Infectious virus titers are then determined through an end point dilution assay (see Baculovirus Expression Vectors, O'Reilly et al. Oxford University Press, New York, 1994).

Quantification of effectiveness of RG-102240-mediated suppression of generation of baculovirus virion through shRNA targeting of the essential virus capsid protein vp80 as described above is carried out by comparing the results of the end point dilution assay with and without RG-102240-mediated expression of shRNA. As summarized in FIG. 7B, RG-102240-mediated suppression of vp80 significantly reduced the levels of budded virus production with bMON14272-pE2 (FIG. 7B, left), while addition of RG-102240 had no statistically significant effect on baculovirus virion production with bMON14272-pFastbac Dual (FIG. 7B, right). bMON14272-pFastbac Dual mimics a prior art system which produce no shRNA that targets vp80. Specifically, virus titers from induced bMON14272-pE2 were reduced by more than three fold relative to uninduced bMON14272-pE2 and, similarly, by more than three fold relative to prior art systems. Since infectious virus particles must be removed from the recombinant protein product for many applications, the decrease in titer of infectious virus particles, especially when conducted at industrial scale, represents a significant improvement in respect of production efficiency and reduction in the cost of goods.

### Example 3

### iVBac2

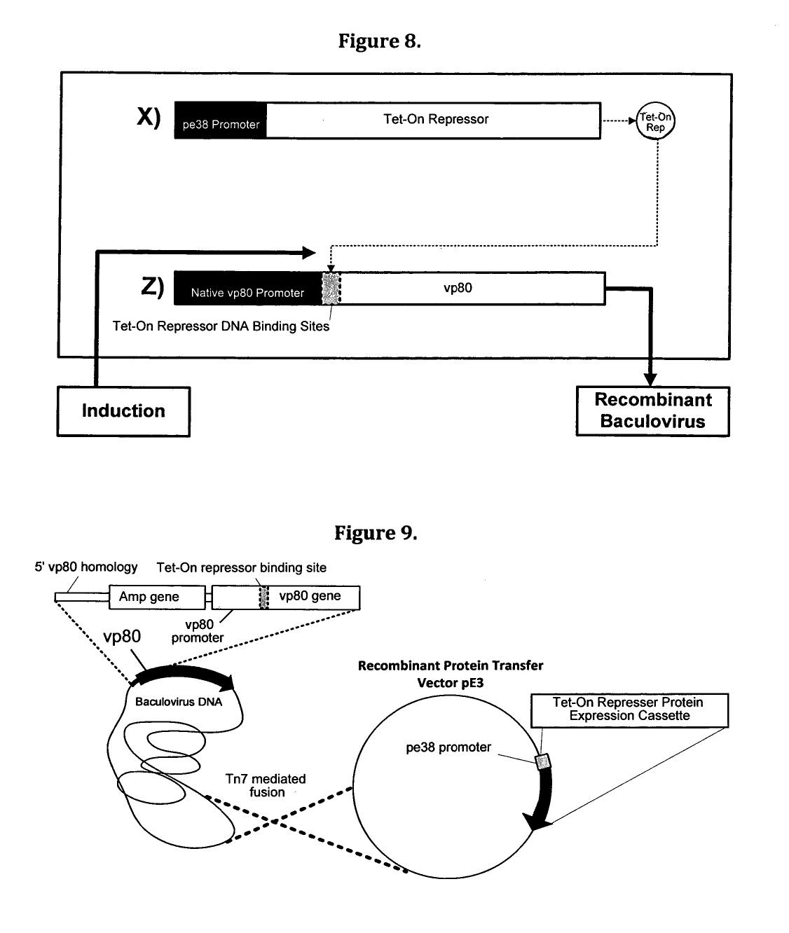

As shown in FIG. 8 an inducible transcriptional repressor protein, the Tet-On repressor (also known as rTetR, here called “Tet-On”; Seq ID No: 12), is the product of expression cassette X. Tet-On was discovered through a 4 amino acid mutation of the native tet repressor which causes Tet-On to bind its DNA recognition sequence with high affinity only in the presence of tetracycline, rather than in the absence of tetracycline as with the native tet repressor protein (Science 268, 1766-9). Expression cassette X is under control of the early baculovirus pe38 promoter (Seq ID No: 2). Expression cassette Z encodes the native vp80 protein (Seq ID No: 8) under control of its native late promoter (Seq ID No: 9). Tet-On has a DNA binding site (transcriptional repressor response element; Seq ID No: 10) just downstream of the vp80 promoter in expression cassette Z. In the absence of tetracycline, Tet-On remains unbound to its DNA binding site in Z. When tetracycline is provided to the system, Tet-On binds to its DNA recognition sequence in Z and thereby represses transcription of the essential baculovirus capsid protein vp80. Baculovirus virion production by baculovirus-infected insect cells in the presence and absence of tetracycline is quantified by measuring virus titers through a virus plaque assay (J. Gen. Virol. 36, 361-364) and PCR (Biotechnology and Bioengineering 108, 1056-1067) or through an end point dilution assay (see Baculovirus Expression Vectors, O'Reilly et al. Oxford University Press, New York, 1994).

The strategy for creation of composite baculovirus containing of the expression cassettes X, and Z is described schematically in FIG. 9. Here Z, the modified vp80 expression cassette containing Tet-On DNA recognition sites just downstream of its promoter, is located on a modified baculovirus DNA derived from bMON14272 (J. Virology 67, 4566-4579). To modify the vp80 promoter in bMON14272 to introduce the Tet-On DNA recognition sequence into the baculovirus genome, baculovirus DNA is modified through ET recombination (Nat. Genet. 20, 123-128) using the protocols described in (Nature Biotechnology 22, 1583-1587).

Briefly, the vector pBAD-ETgamma carrying truncated recE under the arabinose-inducible PBAD promoter and recT under the EM7 promoter is modified by placing the zeocin resistance gene from pPICZA into the FspI and ScaI sites as described for pBADZ-His6Cre yielding pBADZ-ETgamma. This vector is transformed into DH10BAC (Invitrogen) cells harboring the bacmid bMON14272 and the helper plasmid pMON712417 and a kanamycin, tetracyclin and zeocin resistant colony is cultured and 1-arabinose added to 0.1% and the culture is incubated until OD600=0.5. Electro-competent DH10BACET cells are then generated following standard procedures and stored at −80° C.

Next, to integrate the modified vp80 promoter sequence into bMON14272, the linear DNA fragment shown in FIG. 9 (upper left, and Seq ID No: 13) is produced by gene synthesis and 5 μg is electroporated into DH10BACET cells. Transformed cells are grown for 4 h at 37° C. and plated on agar plates containing kanamycin, tetracycline and ampicillin. Bacmid DNA from two single triple-resistant colonies is analyzed by PCR to confirm correct integration. Integrants are made electro-competent according to (Nature Biotechnology 22, 1583-1587) and recombinant protein transfer vector pE3 is introduced into this modified baculovirus DNA as described in the next paragraph.

Cassette X coding for Tet-On is located on transfer vector pE3 (FIG. 9). Transfer vector pE3 is based on pFastbac Dual (Invitrogen) and is constructed as follows. Briefly, the HindIII-KpnI fragment provided in Seq ID No: 14 is prepared by gene synthesis, and is subcloned into HindIII-KpnI digested pFastbac Dual. Composite baculovirus DNA containing expression cassettes X and Z is created by fusing transfer vector pE3 with modified bMON14272 from above. This is carried out by transforming pE3 into the electrocompetent cells prepared as described in the previous paragraph. Tn7 mediated recombination in the competent cells then mediates the fusion of the modified bMON14272 with pE3 as described in (J. Virology 67, 4566-4579).

To create composite baculovirus modified-bMON14272-pE3 and baculovirus modified-bMON14272-pFastBAC Dual (negative control), composite bacmid DNA is isolated from DH10BAC following Tn7 transposition with either pFastbac Dual or pE3, respectively, and transfected into SF21 insect cells according to (Nature Methods 3, 1021-1032). modified-bMON14272-pE3 and modified-bMON14272-pFastbac Dual initial transfection virus is then harvested and used for subsequent baculovirus virion suppression tests.

Baculovirus virion suppression tests are carried out as follows. 10 milliliters of SF21 cells in suspension culture (Nature Methods 3, 1021-1032) at density 0.5×10⁶ cells/ml are infected at multiplicity of infection (M.O.I) of 1.0 with either modified-bMON14272-pE3 or modified-bMON14272-pFastbac Dual initial transfection virus either in the presence, or absence of 5 μg/ml tetracycline. Following 72 hours incubation, cells are centrifuged for 10 minutes at 4000 rpm in a tabletop centrifuge and virus supernatant is harvested. Infectious virus titers are then determined through plaque assay (J. Gen. Virol. 36, 361-364) or an end point dilution assay (see Baculovirus Expression Vectors, O'Reilly et al. Oxford University Press, New York, 1994). The production of infectious virus particles together with non-infectious virus particles and occluded virus particles is quantified by PCR (Biotechnology and Bioengineering 108, 1056-1067) carried out on both infected cells and virus supernatant.

Quantification of effectiveness of tetracycline-mediated suppression of generation of baculovirus virions through Tet-On-mediated repression of transcription of the essential virus capsid protein vp80 is carried out by comparing the results of the end point dilution assay or the plaque assay and PCR with and without tetracycline. It is anticipated that tetracycline-mediated suppression of vp80 transcription will significantly reduce the levels of baculovirus virion production with modified-bMON14272-pE3, while addition of tetracycline will have little or no effect on budded virus production with modified-bMON14272-pFastbac Dual.

### Example 4

### iVBac+iBac

As shown in FIG. 10 an inducible transcriptional repressor protein, the tet repressor protein (tetR; Seq ID No: 1), is the product of expression cassette X/A. Expression cassette X/A is under control of the early baculovirus pe38 promoter (Seq ID No: 2). Expression cassette Y encodes an shRNA, targeting through RNA interference the RNA product of expression cassette Z. To target the baculovirus vp80 gene, the shRNA sequence is designed by use of the following algorithms: Gene Link shRNA Design, (GeneLine), BLOCK-iT™ RNAi Designer (Invitrogen), GenScript siRNA Construct Builder (GeneScript). Other algorithms may be analogously used. The shRNA sequence used in this example is given in Seq ID No: 3 and alternative shRNA sequences may include Seq ID No: 4, Seq ID No: 5, or Seq ID No: 6. Expression cassette Y is under control of the late baculovirus promoter ORF-54 (Seq ID No: 7). Expression cassette Z encodes the native baculovirus capsid protein vp80 (Seq ID No: 8) under control of its native late promoter (Seq ID No: 9). Expression cassette B encodes the native baculovirus very late transcription factor protein vlf-1 (Seq ID No: 21) under control of its native promoter (Seq ID No: 23). Expression cassette C encodes EGFP (U.S. Pat. No. 6,172,188) under control of the baculovirus very late promoter polh (Seq ID No: 15). tetR has DNA binding sites (transcriptional repressor response element; Seq ID No: 10) just downstream of the ORF-54 promoter in expression cassette Y and just downstream of the polh promoter in expression cassette C. In the absence of tetracycline, tetR remains bound to its DNA binding sites in Y and C thereby inhibiting transcription from both expression cassettes. When tetracycline is provided to the system, tetR releases from its DNA binding sites and transcription of shRNA targeting vp80 RNA, and polh-driven recombinant EGFP is induced. Baculovirus virion production by baculovirus-infected insect cells in the presence and absence of tetracycline is quantified by measuring virus titers through a virus plaque assay (J. Gen. Virol. 36, 361-364) and PCR (Biotechnology and Bioengineering 108, 1056-1067) or through an end point dilution assay (see Baculovirus Expression Vectors, O'Reilly et al. Oxford University Press, New York, 1994). EGFP production in the presence and absence of tetracycline is quantified by measuring absorbance of insect cell lysates post infection.

The strategy for creation of composite baculovirus containing the expression cassettes X/A, Y, Z, B and C is described schematically in FIG. 11. Here Z, the native vp80 expression cassette, and B, native vlf-1 are both located on the baculovirus DNA bMON14272 (J. Virology 67, 4566-4579). Cassettes X/A, coding for tetR Y, coding for the shRNA, and C, coding for EGFP are all located on a transfer vector called pE4 (FIG. 11). Transfer vector pE4 is based on pFastbac Dual (Invitrogen) and is constructed as follows. Briefly, the HindIII-KpnI fragment provided in Seq ID No: 16 is prepared by gene synthesis, and is subcloned into HindIII-KpnI digested pFastbac Dual.

Composite baculovirus DNA containing expression cassettes X/A, Y, Z, B and C is created by fusing transfer vector pE4 with bMON14272 (FIG. 11). This is carried out by transforming pE4 into DH10BAC (Invitrogen) E. coli cells harboring the bacmid bMON14272 and the helper plasmid pMON712417. Tn7 mediated recombination in the DH10BAC cells then mediates the fusion of bMON14272 with pE4 as described in (J. Virology 67, 4566-4579).

To create composite baculovirus bMON14272-pE4 and baculovirus bMON14272-pFastBAC Dual (negative control), composite bacmid DNA is isolated from DH10BAC following Tn7 transposition with either pFastbac Dual or pE4, respectively, and transfected into SF21 insect cells according to (Nature Methods 3, 1021-1032). bMON14272-pE4 and bMON14272-pFastbac Dual initial transfection virus is then harvested and used for subsequent baculovirus virion suppression tests, and EGFP expression tests.

Baculovirus virion suppression tests are carried out as follows. 10 milliliters of SF21 cells in suspension culture (Nature Methods 3, 1021-1032) at density 0.5×10⁶ cells/ml are infected at multiplicity of infection (M.O.I) of 1.0 with either bMON14272-pE4 or bMON14272-pFastbac Dual initial transfection virus either in the presence, or absence of 5 μg/ml tetracycline. Following 72 hours incubation, cells are centrifuged for 10 minutes at 4000 rpm in a tabletop centrifuge and virus supernatant is harvested. Infectious virus titers are then determined through plaque assay (J. Gen. Virol. 36, 361-364) an end point dilution assay (see Baculovirus Expression Vectors, O'Reilly et al. Oxford University Press, New York, 1994). The production of infectious virus particles together with non-infectious virus particles and occluded virus particles is quantified by PCR (Biotechnology and Bioengineering 108, 1056-1067) carried out on both infected cells and virus supernatant.

Quantification of effectiveness of tetracycline-mediated suppression of generation of baculovirus virions through shRNA targeting of the essential virus capsid protein vp80 is carried out by comparing the results of the end point dilution assay or the plaque assay and PCR with and without tetracycline-mediated expression of shRNA. It is anticipated that tetracycline-mediated suppression of vp80 will significantly reduce the levels of baculovirus virion production with bMON14272-pE4, while addition of tetracycline will have little or no effect on baculovirus virion production with bMON14272-pFastbac Dual.

EGFP expression tests are carried out by measuring absorbance of cell lysates as described in (Nature Biotechnology 22, 1583-1587). It is anticipated that tetracycline-mediated induction of EGFP expression will significantly increase absorbance of cell lysates around the absorbance maximum of EGFP (490 nm) with bMON14272-pE4, while addition of tetracycline will have little or no effect on the absorbance of cell lysates around the absorbance maximum of EGFP (490 nm) with bMON14272-pFastbac Dual.

As described above, controllable shRNA targeting of VP80 is intended to suppress recombinant baculovirus production. However, it is useful to establish that shRNA production does not substantially and negatively impact recombinant protein production, in this case EGFP. To address this question bMON14272-pE4.1 (as a “wild-type” baculovirus expression system) is carried along in parallel to experiments with bMON14272-pE4 as a positive control for EGFP production. It is desirable that comparable levels of EGFP production are observed from bMON14272-pE4 and bMON14272-pE4.1 both in the presence and absence of tetracycline. If this is not the case, additional shRNA sequences may be examined to find one that does not substantially and negatively impact EGFP production. A person of ordinary technical skill could use this general strategy to test for the effectiveness of shRNAs targeting multiple sequences along VP80, shRNAs targeting sequences along other essential baculovirus capsid protein genes, or combinations of VP80 and other essential baculovirus capsid protein genes.

### Example 5

### iVBac2+iBac

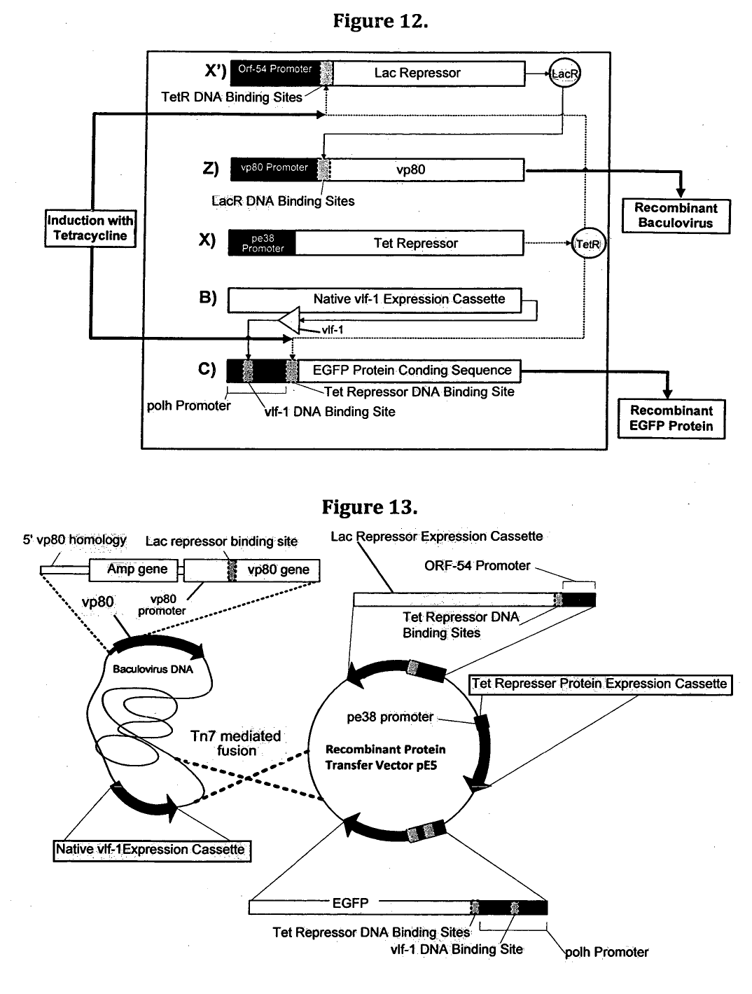

As shown in FIG. 12 an inducible transcriptional repressor protein, the Lac repressor (LacR; PNAS 70, 3576-3580), is the product of expression cassette X′. Expression cassette X′ is under control of the late baculovirus Orf-54 promoter (Seq ID No: 7). Expression cassette Z encodes the native vp80 protein (Seq ID No: 8) under control of its native late promoter (Seq ID No: 9). LacR (second transcriptional repressor protein) has DNA binding sites (second transcriptional repressor response element; Seq ID No: 17) just downstream of the vp80 promoter in expression cassette Z. The tet repressor protein (tetR; Seq ID No: 1), is the product of expression cassette X. Expression cassette X is under control of the early baculovirus pe38 promoter (Seq ID No: 2). Expression cassette B encodes the native baculovirus very late transcription factor protein vlf-1 (Seq ID No: 21) under control of its native promoter (J Virol. 68, 7746-56). Expression cassette C encodes EGFP (U.S. Pat. No. 6,172,188) under control of the baculovirus very late promoter polh (Seq ID No: 15). tetR from expression cassette X has DNA binding sites (transcriptional repressor response element; Seq ID No: 10) just downstream of the Orf-54 promoter in expression cassette X′ and just downstream of the polh promoter in expression cassette C. In the absence of tetracycline, tetR remains bound to its DNA binding sites in X′ and C. When tetracycline is provided to the system, tetR releases from its DNA binding sites in X′ and C, thereby inducing transcription of LacR from X′, and EGFP from C. Tetracycline-induced LacR protein from X′ is thereby available to bind to its recognition sequence in Z, triggering repression of vp80 transcription. Baculovirus virion production by baculovirus-infected insect cells in the presence and absence of tetracycline is quantified by measuring virus titers through a virus plaque assay (J. Gen. Virol. 36, 361-364) and PCR (Biotechnology and Bioengineering 108, 1056-1067) or through an end point dilution assay (see Baculovirus Expression Vectors, O'Reilly et al. Oxford University Press, New York, 1994). EGFP production in the presence and absence of tetracycline is quantified by measuring absorbance of insect cell lysates post infection.

The strategy for creation of composite baculovirus containing of the expression cassettes X, X′, Z, B and C is described schematically in FIG. 13. Cassettes X′, coding for LacR, X, coding for tetR, and C, coding for EGFP are all located on a transfer vector called pE5 (FIG. 13). Transfer vector pE5 is based on pFastbac Dual (Invitrogen) and is constructed as follows. Briefly, the HindIII-KpnI fragment provided in Seq ID No: 18 is prepared by gene synthesis, and is subcloned into HindIII-KpnI digested pFastbac Dual.

Cassettes B, coding for native vlf-1, and Z, coding for the modified vp80 expression cassette containing LacR DNA recognition sites (second transcriptional repressor response element) just downstream of its promoter, are both located on a modified baculovirus DNA derived from bMON14272 (J. Virology 67, 4566-4579). To modify the vp80 promoter in bMON14272 to introduce the LacR DNA recognition sequence into the baculovirus genome, baculovirus DNA is modified through ET recombination (Nat. Genet. 20, 123-128) using the protocols described in (Nature Biotechnology 22, 1583-1587).

Briefly, the vector pBAD-ETgamma carrying truncated recE under the arabinose-inducible PBAD promoter and recT under the EM7 promoter is modified by placing the zeocin resistance gene from pPICZA into the FspI and ScaI sites as described for pBADZ-His6Cre yielding pBADZ-ETgamma. This vector is transformed into DH10BAC (Invitrogen) cells harboring the bacmid bMON14272 and the helper plasmid pMON712417 and a kanamycin, tetracyclin and zeocin resistant colony is cultured and 1-arabinose added to 0.1% and the culture is incubated until OD600=0.5. Electro-competent DH10BACET cells are then generated following standard procedures and stored at −80° C.

Next, to integrate the modified vp80 promoter sequence into bMON14272, the linear DNA fragment shown in FIG. 13 (upper left, and Seq ID No: 19) is produced by gene synthesis and 5 μg is electroporated into DH10BACET cells. Transformed cells are grown for 4 h at 37° C. and plated on agar plates containing kanamycin, tetracycline and ampicillin. Bacmid DNA from two single triple-resistant colonies is analyzed by PCR to confirm correct integration. Integrants are made electro-competent according to (Nature Biotechnology 22, 1583-1587) and recombinant protein transfer vector pE5 is introduced into this modified baculovirus DNA as described in the next paragraph.

Composite baculovirus DNA containing expression cassettes X, X′, Z, B and C is created by fusing transfer vector pE5 with modified bMON14272 from above. This is carried out by transforming pE5 into the electrocompetent cells prepared as described in the previous paragraph. Tn7 mediated recombination in the competent cells then mediates the fusion of the modified bMON14272 with pE5 as described in (J. Virology 67, 4566-4579).

To create composite baculovirus modified-bMON14272-pE5 and baculovirus modified-bMON14272-pFastBAC Dual (negative control), composite bacmid DNA is isolated from DH10BAC following Tn7 transposition with either pFastbac Dual or pE5, respectively, and transfected into SF21 insect cells according to (Nature Methods 3, 1021-1032). Modified-bMON14272-pE5 and modified-bMON14272-pFastbac Dual initial transfection virus is then harvested and used for subsequent baculovirus virion suppression tests.

Baculovirus virion suppression tests are carried out as follows. 10 milliliters of SF21 cells in suspension culture (Nature Methods 3, 1021-1032) at density 0.5×10⁶ cells/ml are infected at multiplicity of infection (M.O.I) of 1.0 with either modified-bMON14272-pE5 or modified-bMON14272-pFastbac Dual initial transfection virus either in the presence, or absence of 5 μg/ml tetracycline. Following 72 hours incubation, cells are centrifuged for 10 minutes at 4000 rpm in a tabletop centrifuge and virus supernatant is harvested. Infectious virus titers are then determined through plaque assay (J. Gen. Virol. 36, 361-364) an end point dilution assay (see Baculovirus Expression Vectors, O'Reilly et al. Oxford University Press, New York, 1994). The production of infectious virus particles together with non-infectious virus particles and occluded virus particles is quantified by PCR (Biotechnology and Bioengineering 108, 1056-1067) carried out on both infected cells and virus supernatant.

Quantification of effectiveness of tetracycline-mediated suppression of generation of baculovirus virions through Tet-On induced suppression of transcription of the essential virus capsid protein vp80 is carried out by comparing the results of the end point dilution assay or the plaque assay and PCR with and without tetracycline. It is anticipated that tetracycline-mediated suppression of vp80 transcription will significantly reduce the levels of budded virus production with modified-bMON14272-pE5, while addition of tetracycline will have little or no effect on baculovirus virion production with modified-bMON14272-pFastbac Dual.

EGFP expression tests are carried out by measuring absorbance of cell lysates as described in (Nature Biotechnology 22, 1583-1587). It is anticipated that tetracycline-induced induction of EGFP expression will significantly increase absorbance of cell lysates around the absorbance maximum of EGFP (490 nm) with bMON14272-pE5, while addition of tetracycline will have little or no effect on the absorbance of cell lysates around the absorbance maximum of EGFP (490 nm) with bMON14272-pFastbac Dual.

1. A method for the production of a recombinant protein in insect cells, said method comprising the steps:
(a) providing insect cells comprising a baculovirus expression system containing a nucleotide sequence encoding said recombinant protein;
(b) maintaining during a scale up phase said insect cells of step (a) under a first condition such that said baculovirus expression system produces infectious baculovirus virions;
(c) maintaining during a recombinant protein production phase said insect cells of step (b) under a second condition such that production of baculovirus virions is suppressed; and
(d) harvesting said recombinant protein expressed by said baculovirus expression system,
wherein the baculovirus expression system comprises an inducible expression control system that:
suppresses expression of at least one gene product essential for baculovirus virion assembly under said second condition during production of said recombinant protein; and
allows expression of said at least one gene product essential for baculovirus virion assembly under said first condition.

2. The method of claim 1, wherein additionally under said first condition the expression of said recombinant protein is suppressed.

3. The method of claim 1, wherein:
In step (b) said first condition suppresses expression of said recombinant protein in addition to allowing production of said baculovirus virions; and
In step (c) said second condition allows expression of said recombinant protein in addition to suppressing production of said baculovirus virions.

4. The method of claim 1, wherein the at least one gene product essential for baculovirus virion assembly is selected from the group consisting of vp80, vp39, vp1054, gp64, p74, p24 and p6.9.

5. The method of claim 1, wherein said baculovirus expression system comprises:
at least one expression cassette X comprising a promoter and an open reading frame coding for a controllable transcriptional modulator protein;
at least one expression cassette Y comprising a promoter and a nucleotide sequence coding for an RNAi effector targeting said at least one gene product essential for baculovirus virion assembly;
at least one expression cassette Z comprising a promoter and an open reading frame coding for said gene product essential for baculovirus virion assembly; and
at least one transcriptional modulator response element in expression cassette Y,
wherein said controllable transcriptional modulator protein reversibly interacts with said transcriptional modulator response element(s) in said first condition, and interacts differently in said second condition, thereby modulating transcription of expression cassette Y.

6. (canceled)

7. (canceled)

8. The method of claim 5, wherein the baculovirus expression system comprises:
at least one expression cassette X comprising a promoter and an open reading frame coding for a controllable transcriptional repressor protein;
at least one expression cassette Y comprising a promoter, a transcriptional repressor response element and a nucleotide sequence coding for an RNAi effector targeting said at least one gene product essential for baculovirus virion assembly; and
at least one expression cassette Z comprising a promoter and an open reading frame coding for said gene product essential for baculovirus virion assembly,
wherein:
said first condition maintained during scale up phase is repressive, thereby repressing transcription of said RNAi effector and allowing expression of said gene product essential for baculovirus virion assembly, and
said second condition maintained during recombinant protein production phase is non-repressive, thereby inducing transcription of said RNAi effector and suppressing expression of said gene product essential for baculovirus virion assembly; and
further wherein:
the promoter in expression cassette X has an earlier time of onset of gene expression than the promoter in expression cassette Y;
the promoter in expression cassette Y has an earlier or simultaneous time of onset of gene expression than the promoter in expression cassette Z; and optionally
the promoter in expression cassette Z is the native promoter of said gene product essential for baculovirus virion assembly.

9. (canceled)

10. The method of claim 5, wherein the baculovirus expression system comprises:
at least one expression cassette X comprising a promoter and an open reading frame coding for a controllable transcriptional activator protein;
at least one expression cassette Y comprising a minimal promoter, a transcriptional activator response element and a nucleotide sequence coding for an RNAi effector targeting said at least one gene product essential for baculovirus virion assembly; and
at least one expression cassette Z comprising a promoter and an open reading frame coding for said gene product essential for baculovirus virion assembly,
wherein:
said first condition maintained during scale up phase is non-activating, thereby repressing transcription of said RNAi effector and allowing expression of said gene product essential for baculovirus virion assembly, and
said second condition maintained during recombinant protein production phase is activating, thereby inducing transcription of said RNAi effector and suppressing expression of said gene product essential for baculovirus virion assembly; and
further wherein:
the promoter in expression cassette X has an earlier time of onset of gene expression than the promoter in expression cassette Y;
the promoter in expression cassette Y has an earlier or simultaneous time of onset of gene expression than the promoter in expression cassette Z; and optionally
the promoter in expression cassette Z is the native promoter of said gene product essential for baculovirus virion assembly.

11. (canceled)

12. The method of claim 2, wherein said baculovirus expression system comprises:
at least one expression cassette A comprising a promoter and an open reading frame coding for a controllable transcriptional modulator protein,
at least one expression cassette B comprising a promoter and an open reading frame coding for a factor which regulates transcriptional activity of a baculovirus late and/or very late promoter,
at least one expression cassette C comprising an open reading frame coding for said recombinant protein under the control of a baculovirus late and/or very late promoter responsive to the factor expressed by expression cassette B, and
at least one transcription modulator response element in expression cassette C,
wherein said controllable transcriptional modulator protein expressed by expression cassette A reversibly interacts with said transcriptional modulator response element(s) in said first condition, and interacts differently in said second condition, thereby modulating the transcription of expression cassette C.

13. The method of claim 12, wherein said baculovirus expression system comprises:
at least one expression cassette X comprising a promoter that controls expression of a controllable transcriptional repressor protein;
at least one expression cassette Y comprising a promoter, a transcriptional repressor response element and a nucleotide sequence coding for an RNAi effector targeting said at least one gene product essential for baculovirus virion assembly;
at least one expression cassette Z comprising a promoter and an open reading frame coding for said gene product essential for baculovirus virion assembly,
at least one expression cassette A, comprising a promoter and an open reading frame coding for a controllable transcriptional repressor protein,
at least one expression cassette B comprising a promoter and an open reading frame coding for a factor which regulates transcriptional activity of a baculovirus late and/or very late promoter,
at least one expression cassette C comprising an open reading frame coding for said recombinant protein under the control of a baculovirus late and/or very late promoter responsive to the factor expressed by expression cassette B, and
at least one transcriptional repressor response element in expression cassette C,
wherein:
said first condition maintained during scale up phase is repressive, thereby:
repressing transcription of said RNAi effector and allowing expression of said gene product essential for baculovirus virion assembly, and
repressing expression of said recombinant protein; and
said second condition maintained during recombinant protein production phase is non-repressive, thereby:
inducing transcription of said RNAi effector and suppressing expression of said gene product essential for baculovirus virion assembly, and
inducing expression of said recombinant protein.

14. The method of claim 12, wherein said baculovirus late and/or very late promoter in expression cassette C is a baculovirus very late promoter.

15. The method of claim 12, wherein said factor encoded by expression cassette B is vlf-1 of Seq ID No: 21 or a transcriptionally functional homolog thereof.

16. (canceled)

17. The method of claim 13, characterised in that:
the expression cassette X and expression cassette A are coding for the same controllable transcriptional modulator protein and are either independent cassettes or one single expression cassette X/A; and
said controllable transcriptional modulator protein expressed by expression cassette X/A reversibly interacts with said transcriptional modulator response elements in said first condition, and interacts differently in said second condition, thereby modulating the transcription of both expression cassette Y and expression cassette C.

18. The method claim 1, wherein said baculovirus expression system comprises:
at least one expression cassette X comprising a promoter and an open reading frame coding for a controllable transcriptional repressor protein;
at least one expression cassette Z comprising a promoter and an open reading frame coding for a gene product essential for baculovirus virion assembly; and
at least one transcriptional repressor response element in expression cassette Z, wherein said controllable transcriptional repressor protein reversibly interacts with its transcriptional repressor response element(s) in said first condition, and interacts differently in said second condition, thereby directly or indirectly modulating the transcription of expression cassette Z, and
further wherein:
said first condition maintained during scale up phase allows expression of said gene product essential for baculovirus virion assembly; and
said second condition maintained during recombinant protein production phase suppresses expression of said gene product essential for baculovirus virion assembly.

19. (canceled)

20. The method of claim 18, wherein said baculovirus expression system comprises:
at least one expression cassette X comprising a promoter and an open reading frame coding for a controllable transcriptional repressor protein;
at least one expression cassette X′ comprising a promoter and an open reading frame coding for a second transcriptional repressor protein, different to the controllable transcriptional repressor protein encoded by expression cassette X;
at least one expression cassette Z comprising a promoter and an open reading frame coding for a gene product essential for baculovirus virion assembly,
at least one expression cassette B comprising a promoter and an open reading frame coding for a factor which regulates transcriptional activity of a baculovirus late and/or very late promoter,
at least one expression cassette C comprising an open reading frame coding for the recombinant protein under the control of a baculovirus late and/or very late promoter responsive to the factor expressed by expression cassette B,
at least one transcriptional repressor response element in expression cassette X′ and expression cassette C responsive to said controllable transcriptional repressor protein encoded by expression cassette X, and
at least one second transcriptional repressor response element in expression cassette Z responsive to said second transcriptional repressor protein encoded by expression cassette X′,
wherein said controllable transcriptional repressor protein encoded by expression cassette X reversibly interacts with said transcriptional repressor response element in expression cassette X′ and in expression cassette C, and interacts differently in said second condition, thereby modulating the transcription of the second transcriptional repressor protein encoded by expression cassette X′ and modulating the transcription of the recombinant protein encoded by expression cassette C, and
further wherein:
said first condition maintained during scale up phase is repressive, thereby:
repressing expression of said second transcriptional repressor protein encoded by expression cassette X′, thereby allowing expression of said gene product essential for baculovirus virion assembly, and
repressing expression of said recombinant protein; and
said second condition maintained during recombinant protein production phase is non-repressing, thereby:
inducing expression of said second transcriptional repressor protein encoded by expression cassette X′, thereby suppressing expression of said gene product essential for baculovirus virion assembly, and
inducing expression of said recombinant protein.

21. The method of claim 20 wherein:
said baculovirus late and/or very late promoter in expression cassette C is a baculovirus very late promoter;
said factor encoded by expression cassette B is vlf-1 of Seq ID No: 8 or a transcriptionally functional homolog thereof; and/or
the second transcriptional repressor protein encoded by expression cassette X′ is a controllable transcriptional repressor protein or is a constitutive transcriptional repressor protein; preferably the second transcriptional repressor protein encoded by expression cassette X′ is a lac repressor.

22. (canceled)

23. (canceled)

24. (canceled)

25. The method of claim 1, wherein suppressing said expression of said at least one gene product essential for baculovirus virion assembly does not substantially affect late and/or very late recombinant protein expression from said baculovirus expression system in comparison to late and/or very late expression from said baculovirus expression system without suppressing said expression of said at least one gene product essential for baculovirus virion assembly.

26. The method of claim 1, wherein said recombinant protein harvested in step (d) contains reduced amounts of baculovirus virions compared to recombinant protein harvested from a method without suppressing expression of said at least one gene product essential for baculovirus virion assembly during recombinant protein production phase.

27. The method of claim 1, wherein said recombinant protein harvested in step (d) is separated and/or purified from contaminating components.

28. The method claim 1, wherein the recombinant protein is a biopharmaceutical virus like particle or a biopharmaceutical.

29. The method of claim 1, wherein said baculovirus expression system is introduced into said insect cells by transfection or infection prior to step (a).

30. A baculovirus expression system as defined in claim 5.

31. A baculovirus transfer vector comprising expression cassette Y of the baculovirus expression system as defined in claim 5.

32. The baculovirus transfer vector of claim 31, further comprising expression cassette(s) X comprising a promoter and an open reading frame coding for a controllable transcriptional modulator protein; and/or
expression cassette C comprising an open reading frame coding for said recombinant protein under the control of a baculovirus late and/or very late promoter responsive to the factor expressed by expression cassette B, wherein expression cassette B comprises a promoter and an open reading frame coding for a factor which regulates transcriptional activity of a baculovirus late and/or very late promoter.

33. A composite baculovirus DNA comprising the baculovirus expression system of claim 30.

34. An insect cell comprising the baculovirus expression system of claim 5.

35. The method of claim 1, wherein the baculovirus expression system is derived from a nuclear polyhedosis virus (NPV).

36. (canceled)

37. A kit for repressing production of baculovirus virions during recombinant protein production phase in insect cells comprising:
(i) at least one expression cassette C⁽⁻⁾ containing a baculovirus late and/or very late promoter, wherein said expression cassette is intended for expressing a recombinant protein under the control of said promoter,
(ii) at least one expression cassette Y as defined in claim 13; and
(iii) expression cassette X and/or A as defined in claim 13.

38. The kit of claim 37, wherein at least one of expression cassette C⁽⁻⁾ expression cassette Y and expression cassette X and/or A is comprised on a transfer vector.

39. The kit of claim 37, wherein the expression cassette C⁽⁻⁾ further comprises a transcriptional modulator response element.

40. The kit of claim 37, wherein expression cassette C⁽⁻⁾ further contains an open reading frame for expressing said recombinant protein under the control of said promoter.

41. A recombinant nucleic acid comprising at least expression cassette Z as defined in claim 18.

42. (canceled)

43. (canceled)

44. A composition comprising at least the recombinant nucleic acid of claim 41.

45. The composition of claim 44, further comprising at least one recombinant nucleic acid selected from: (i) expression cassette X, comprising a promoter and an open reading frame coding for a controllable transcriptional modulator protein and/or expression cassette A, comprising a promoter and an open reading frame coding for a controllable transcriptional repressor protein; (ii) expression cassette X, comprising a promoter and an open reading frame coding for a controllable transcriptional modulator protein and/or X′, comprising a promoter and an open reading frame coding for a second transcriptional repressor protein, different to the controllable transcriptional repressor protein encoded by expression cassette X; or (iii) expression cassette C, comprising an open reading frame coding for said recombinant protein under the control of a baculovirus late and/or very late promoter responsive to the factor expressed by expression cassette B, comprising a promoter and an open reading frame coding for a factor which regulates transcriptional activity of a baculovirus late and/or very late promoter.

46. The composition of claim 44 in a cell selected from the group of an insect cell, a bacterial cell and a mammalian cell.

47. A kit comprising:
(i) The vector of claim 43; and
(ii) At least one other component for the construction and/or use of a baculovirus expression system to repress production of baculovirus virions during production of a recombinant protein, preferably such as any component defined in any one of the preceding claims.

48. (canceled)

49. (canceled)


SEQUENCE LISTING (23 sequences)

SEQ ID NO: 1 - protein, 207 aa, Escherichia coli
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGS

SEQ ID NO: 2 - DNA, 125 bp, Autographa californica
tatcacaggctgcagtttctgttatctgtcccccactcaggcgtgcagctataaaagcaggcactcaccaactcgtaagcacagttcgttgtgaagtgaacacggagagcctgccaataagcaaa

SEQ ID NO: 3 - DNA, 56 bp, Artificial Sequence
  Description of Artificial Sequence Synthetic shRNA
ttagaggtaatgttggaagcgcttactcgagaaagcgcttccaacattacctctaa

SEQ ID NO: 4 - DNA, 62 bp, Artificial Sequence
  Description of Artificial Sequence Synthetic shRNA
attagaatttagcaaggacattgtcaaactcgagattgacaatgtccttgctaaattctaat

SEQ ID NO: 5 - DNA, 58 bp, Artificial Sequence
  Description of Artificial Sequence Synthetic shRNA
tgaccagcatggtgttggctgtaaaactcgagatttacagccaacaccatgctggtca

SEQ ID NO: 6 - DNA, 58 bp, Artificial Sequence
  Description of Artificial Sequence Synthetic shRNA
accaggaagattgtgtgatactgaaactcgagattcagtatcacacaatcttcctggt

SEQ ID NO: 7 - DNA, 161 bp, Autographa californica
aacgcgttacacaggaagacgccctggacattgaactcgattttaggcatttttttaaaatgcaatcatgacgaacgtatggttcgcgacggacgtcaacctgatcaattgtgtactgaaagataatttatttttgatagataataattacattattttaa

SEQ ID NO: 8 - protein, 691 aa, Autographa californica
MNDSNSLLITRLAAQILSRNMQTVDVIVDDKTLSLEEKIDTLTSMVLAVNSPPQSPPRVTSSDLAASIIKNNSKMVGNDFEMRYNVLRMAVVFVKHYPKYYNETTAGLVAEIESNLLQYQNYVNQGNYQNIEGYDSLLNKAEECYVKIDRLFKESIKKIMDDTEAFEREQEAERLRAEQTAANALLERRAQTSADDVVNRADANIPTAFSDPLPGPSAPRYMYESSESDTYMETARRTAEHYTDQDKDYNAAYTADEYNSLVKTVLLRLIEKALATLKNRLHITTIDQLKKFRDYLNSDADAGEFQIFLNQEDCVILKNLSNLASKFFNVRCVADTLEVMLEALRNNIELVQPESDAVRRIVIKMTQEIKDSSTPLYNIAMYKSDYDAIKNKNIKTLFDLYNDRLPINFLDTSATSPVRKTSGKRSAEDDLLPTRSSKRANRPEINVISSEDEQEDDDVEDVDYEKESKRRKLEDEDFLKLKALEFSKDIVNEKLQKIIVVTDGMKRLYEYCNCKNSLETLPSAANYGSLLKRLNLYNLDHIEMNVNFYELLFPLTLYNDNDNSDKTLSHQLVNYIFLASNYFQNCAKNFNYMRETFNVFGPFKQIDFMVMFVIKFNFLCDMRNFAKLIDELVPNKQPNMRIHSVLVMRDKIVKLAFSNLQFQTFSKKDKSRNTKHLQRLIMLMNANYNVI

SEQ ID NO: 9 - DNA, 163 bp, Autographa californica
gctttggttgaaatattttgaaaataaaaaacacaacgaatcgatttcatcttgtaacaattgcgcttcaaaacacacgttttcaaagcggtcgtaaatgttaaaccttaaactgtattgtaatctgtaagcgcacatggtgcattcgatataaccttataat

SEQ ID NO: 10 - DNA, 55 bp, Artificial Sequence
  Description of Artificial Sequence Synthetic tetR response element
actctatcagtgatagagttattttaccactccctatcagtgatagagaaaaagc

SEQ ID NO: 11 - DNA, 1240 bp, Artificial Sequence
  Description of Artificial Sequence Synthetic HindIII-KpnI fragment
ggtaccttagaggtaatgttggaagcgcttactcgagaaagcgcttccaacattacctctaaactctatcagtgatagagttattttaccactccctatcagtgatagagaaaaagcttaaaataatgtaattattatctatcaaaaataaattatctttcagtacacaattgatcaggttgacgtccgtcgcgaaccatacgttcgtcatgattgcattttaaaaaaatgcctaaaatcgagttcaatgtccagggcgtcttcctgtgtaacgcgttcaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtatcacaggctgcagtttctgttatctgtcccccactcaggcgtgcagctataaaagcaggcactcaccaactcgtaagcacagttcgttgtgaagtgaacacggagagcctgccaataagcaaaatgtcccgtctggacaagtccaaggtgatcaactccgctctggagctgctgaacgaggtgggtatcgagggtctgaccacccgtaagctggctcagaagctgggtgtggagcagcccaccctgtactggcacgtgaagaacaagcgtgctctgctggacgctctggctatcgagatgctggaccgtcaccacacccacttctgccccctggagggtgagtcctggcaggacttcctgcgtaacaacgctaagtccttccgttgcgctctgctgtcccaccgtgacggtgctaaggtgcacctgggcacccgtcccaccgagaagcagtacgagaccctggagaaccaactggctttcctgtgccagcagggtttctccctggagaacgctctgtacgctctgtccgctgtgggtcacttcaccctgggttgcgtgctggaggaccaggagcaccaggtggctaaggaggagcgtgagacccccaccaccgactccatgccccccctgctgcgtcaggctatcgagctgttcgaccaccagggtgctgagcccgctttcctgttcggtctggagctgatcatctgcggtctggagaagcaactgaagtgcgagtccggttccaagctt

SEQ ID NO: 12 - protein, 206 aa, Artificial Sequence
  Description of Artificial Sequence Tet-On repressor
MSRLDKSKVINSALELLNEVGIEGLTTRKLAQKLGVEQPTLYWHVKNKRALLDALAIEMLDRHHTHFCPLKGESWQDFLRNNAKSFRCALLSHRNGAKVHSDTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESG

SEQ ID NO: 13 - DNA, 1633 bp, Artificial Sequence
  Description of Artificial Sequence Modified vp80 promoter
ggtaccgctttggttgaaatattttgaaaataaaaaacacaacgaacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcatcgatttcatcttgtaacaattgcgcttcaaaacacacgttttcaaagcggtcgtaaatgttaaaccttaaactgtattgtaatctgtaagcgcacatggtgcattcgatataaccttataatactctatcagtgatagagttattttaccactccctatcagtgatagagaaaaagcatgaacgattccaattctctgttgattacgcgtttggcagcgcaaatactgtccagaaacatgcaaacggtggatgtgattgttgacgacaaaacgctcagtttggaagaaaaaatagacacgttgaccagcatggtgttggctgtaaatagcccgccgcaatcgccgccgcgggtaacatccagcgacctggccgctaatagaagctt

SEQ ID NO: 14 - DNA, 755 bp, Artificial Sequence
  Description of Artificial Sequence Synthetic HindIII- -KpnI fragment
ggtacctatcacaggctgcagtttctgttatctgtcccccactcaggcgtgcagctataaaagcaggcactcaccaactcgtaagcacagttcgttgtgaagtgaacacggagagcctgccaataagcaaaatgagccgcctggataaaagcaaagtgattaacagcgcgctggaactgctgaacgaagtgggcattgaaggcctgaccacccgcaaactggcgcagaaactgggcgtggaacagccgaccctgtattggcatgtgaaaaacaaacgcgcgctgctggatgcgctggcgattgaaatgctggatcgccatcatacccatttttgcccgctgaaaggcgaaagctggcaggattttctgcgcaacaacgcgaaaagctttcgctgcgcgctgctgagccatcgcaacggcgcgaaagtgcatagcgatacccgcccgaccgaaaaacagtatgaaaccctggaaaaccagctggcgtttctgtgccagcagggctttagcctggaaaacgcgctgtatgcgctgagcgcggtgggccattttaccctgggctgcgtgctggaagatcaggaacatcaggtggcgaaagaagaacgcgaaaccccgaccaccgatagcatgccgccgctgctgcgccaggcgattgaactgtttgatcatcagggcgcggaaccggcgtttctgtttggcctggaactgattatttgcggcctggaaaaacagctgaaatgcgaaagcggctaatag

SEQ ID NO: 15 - DNA, 129 bp, Autographa californica
atcatggagataattaaaatgataaccatctcgcaaataaataagtattttactgttttcgtaacagttttgtaataaaaaaacctataaatattccggattattcataccgtcccaccatcgggcgcg

SEQ ID NO: 16 - DNA, 2228 bp, Artificial Sequence
  Description of Artificial Sequence Synthetic HindIII -KpnI fragment
ggtaccttagaggtaatgttggaagcgcttactcgagaaagcgcttccaacattacctctaaactctatcagtgatagagttattttaccactccctatcagtgatagagaaaaagcttaaaataatgtaattattatctatcaaaaataaattatctttcagtacacaattgatcaggttgacgtccgtcgcgaaccatacgttcgtcatgattgcattttaaaaaaatgcctaaaatcgagttcaatgtccagggcgtcttcctgtgtaacgcgttcaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtatcacaggctgcagtttctgttatctgtcccccactcaggcgtgcagctataaaagcaggcactcaccaactcgtaagcacagttcgttgtgaagtgaacacggagagcctgccaataagcaaaatgtcccgtctggacaagtccaaggtgatcaactccgctctggagctgctgaacgaggtgggtatcgagggtctgaccacccgtaagctggctcagaagctgggtgtggagcagcccaccctgtactggcacgtgaagaacaagcgtgctctgctggacgctctggctatcgagatgctggaccgtcaccacacccacttctgccccctggagggtgagtcctggcaggacttcctgcgtaacaacgctaagtccttccgttgcgctctgctgtcccaccgtgacggtgctaaggtgcacctgggcacccgtcccaccgagaagcagtacgagaccctggagaaccaactggctttcctgtgccagcagggtttctccctggagaacgctctgtacgctctgtccgctgtgggtcacttcaccctgggttgcgtgctggaggaccaggagcaccaggtggctaaggaggagcgtgagacccccaccaccgactccatgccccccctgctgcgtcaggctatcgagctgttcgaccaccagggtgctgagcccgctttcctgttcggtctggagctgatcatctgcggtctggagaagcaactgaagtgcgagtccggttcctaatagatcatggagataattaaaatgataaccatctcgcaaataaataagtattttactgttttcgtaacagttttgtaataaaaaaacctataaatattccggattattcataccgtcccaccatcgggcgcgactctatcagtgatagagttattttaccactccctatcagtgatagagaaaaagcatggtgtccaagggtgaggagctgttcaccggtgtggtgcccatcctggtggagctggacggtgacgtgaacggtcacaagttctccgtgtccggtgagggtgagggtgacgctacctacggtaagctgaccctgaagttcatctgcaccaccggtaagctgcccgtgccctggcccaccctggtgaccaccctgacctacggtgtgcagtgcttctcccgttaccccgaccacatgaagcagcacgacttcttcaagtccgctatgcccgagggttacgtgcaggagcgtaccatcttcttcaaggacgacggtaactacaagacccgtgctgaggtgaagttcgagggtgacaccctggtgaaccgtatcgagctgaagggtatcgacttcaaggaggacggtaacatcctgggtcacaagctggagtacaactacaactcccacaacgtgtacatcatggctgacaagcagaagaacggtatcaaggtgaacttcaagatccgtcacaacatcgaggacggttccgtgcagctcgctgaccactaccagcagaacacccccatcggtgacggtcccgtgctgctgcccgacaaccactacctgtccacccagtccgctctgtccaaggaccccaacgagaagcgtgaccacatggtgctgctggagttcgtgaccgctgctggtatcaccctgggtatggacgagctgtacaagtccggtctgcgttcccgtgctcaggcttccaactccgctgtggacggtacggctggtcccggttccaccggttcccgttaaaagctt

SEQ ID NO: 17 - DNA, 35 bp, Escherichia coli
tgtgtggaattgtgagcggataacaatttcacaca

SEQ ID NO: 18 - DNA, 3267 bp, Artificial Sequence
  Description of Artificial Sequence Synthetic HindIII -KpnI fragment
ggtaccctattactggccgctttccaggcggctcacctggcgcgccagctgcatcaggctatccgccagcgcgcgcgggctcgcggtctgggtgttcggcggcagggtggttttgcgtttcaccaggctcaccggcagcagctggttgcctttcaccgcctggccctggctcagctgcagcaggcgatccacgctggtctggcccagcaggcgaaaatcctgtttaatggtggtcagcggcggaatatagcagctgctatcttcggtatcatcatagcccaccacgctaatatccgcgcccacgcgcaggccgctttcggtaatcgcgcgcatcgcgcccagcgccatctgatcgttcgccaccagcatcgcggtcggcacaatgccttcgttcagcatctgcatggtctgctgaaagccgctcatcgcgctccaatcgccttcgcgttccgcaatcggctgaatctggttgcgggtcagatatttatgccagcccgccaggcgcaggcgcgcgctcacgctgctcagcgggcccgccagcagcgcaatctgctgatggcccagcgccaccagatgttccacgcccaggcgggtgccatcttcatggctaaaaataatgctgttaatcggggtctgatcgctcacatccagaaacagcgccggcacgttcgcgcacgccgcttccaccgcaatcgcatcctgatcatccagcggatagttaataatcaggccgctcacgcgctgcgccagcaggttatgcaccgccgctttgcacgcttccacgccgctgcgttccaccatgctcaccaccacgctcgcgcccagctgatccgcgcggcttttaatcgccgccacaatctggctcggcgcatgcagcgccaggctgctggtcgccacgccaatcagcaggctctgtttgcccgccagctgctgcgccacgcggttcggaatatagttcagttccgccatcgccgcttccactttttcgcgggttttcgcgctcacatggcacgcctggttcaccacgcggctcacggtctgatagctcacgcccgcatattccgccacatcatacagggtcaccggtttcacgttcaccatactctatcagtgatagagttattttaccactccctatcagtgatagagaaaaagcttaaaataatgtaattattatctatcaaaaataaattatctttcagtacacaattgatcaggttgacgtccgtcgcgaaccatacgttcgtcatgattgcattttaaaaaaatgcctaaaatcgagttcaatgtccagggcgtcttcctgtgtaacgcgttcaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtatcacaggctgcagtttctgttatctgtcccccactcaggcgtgcagctataaaagcaggcactcaccaactcgtaagcacagttcgttgtgaagtgaacacggagagcctgccaataagcaaaatgtcccgtctggacaagtccaaggtgatcaactccgctctggagctgctgaacgaggtgggtatcgagggtctgaccacccgtaagctggctcagaagctgggtgtggagcagcccaccctgtactggcacgtgaagaacaagcgtgctctgctggacgctctggctatcgagatgctggaccgtcaccacacccacttctgccccctggagggtgagtcctggcaggacttcctgcgtaacaacgctaagtccttccgttgcgctctgctgtcccaccgtgacggtgctaaggtgcacctgggcacccgtcccaccgagaagcagtacgagaccctggagaaccaactggctttcctgtgccagcagggtttctccctggagaacgctctgtacgctctgtccgctgtgggtcacttcaccctgggttgcgtgctggaggaccaggagcaccaggtggctaaggaggagcgtgagacccccaccaccgactccatgccccccctgctgcgtcaggctatcgagctgttcgaccaccagggtgctgagcccgctttcctgttcggtctggagctgatcatctgcggtctggagaagcaactgaagtgcgagtccggttcctaatagatcatggagataattaaaatgataaccatctcgcaaataaataagtattttactgttttcgtaacagttttgtaataaaaaaacctataaatattccggattattcataccgtcccaccatcgggcgcgactctatcagtgatagagttattttaccactccctatcagtgatagagaaaaagcatggtgtccaagggtgaggagctgttcaccggtgtggtgcccatcctggtggagctggacggtgacgtgaacggtcacaagttctccgtgtccggtgagggtgagggtgacgctacctacggtaagctgaccctgaagttcatctgcaccaccggtaagctgcccgtgccctggcccaccctggtgaccaccctgacctacggtgtgcagtgcttctcccgttaccccgaccacatgaagcagcacgacttcttcaagtccgctatgcccgagggttacgtgcaggagcgtaccatcttcttcaaggacgacggtaactacaagacccgtgctgaggtgaagttcgagggtgacaccctggtgaaccgtatcgagctgaagggtatcgacttcaaggaggacggtaacatcctgggtcacaagctggagtacaactacaactcccacaacgtgtacatcatggctgacaagcagaagaacggtatcaaggtgaacttcaagatccgtcacaacatcgaggacggttccgtgcagctcgctgaccactaccagcagaacacccccatcggtgacggtcccgtgctgctgcccgacaaccactacctgtccacccagtccgctctgtccaaggaccccaacgagaagcgtgaccacatggtgctgctggagttcgtgaccgctgctggtatcaccctgggtatggacgagctgtacaagtccggtctgcgttcccgtgctcaggcttccaactccgctgtggacggtacggctggtcccggttccaccggttcccgttaaaagctt

SEQ ID NO: 19 - DNA, 1644 bp, Artificial Sequence
  Description of Artificial Sequence Modified vp80 promoter
ggtaccgctttggttgaaatattttgaaaataaaaaacacaacgaacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcatcgatttcatcttgtaacaattgcgcttcaaaacacacgttttcaaagcggtcgtaaatgttaaaccttaaactgtattgtaatctgtaagcgcacatggtgcattcgatataaccttataattgtgtggaattgtgagcggataacaatttcacacaaatgtgtggaattgtgagcggataacaatttcacacaatgaacgattccaattctctgttgattacgcgtttggcagcgcaaatactgtccagaaacatgcaaacggtggatgtgattgttgacgacaaaacgctcagtttggaagaaaaaatagacacgttgaccagcatggtgttggctgtaaatagcccgccgcaatcgccgccgcgggtaacatccagcgacctggccgcaagctt

SEQ ID NO: 20 - DNA, 123 bp, Autographa californica
tatacggacctttaattcacccaacacaatatattatagttaaataagaattattatcaaatcatttgtatattaattaaaatactatactgtaaattacattttatttacaatcactcgacg

SEQ ID NO: 21 - protein, 379 aa, Autographa californica
MNGFNVRNENNFNSWKIKIQSAPRFESVFDLATDRQRCTPDEVKNNSLWSKYMFPKPFAPTTLKSYKSRFIKIVYCSVDDVHLEDMSYSLDKEFDSIENQTLLIDPQELCRRMLELRSVTKETLQLTINFYTNMMNLPEYKIPRMVMLPRDKELKNIREKEKNLMLKNVIDTILNFINDKIKMLNSDYVHDRGLIRGAIVFCIMLGTGMRINEARQLSVDDLNVLIKRGKLHSDTINLKRKRSRNNTLNNIKMKPLELAREIYSRNPTILQISKNTSTPFKDFRRLLEESGVEMERPRSNMIRHYLSSNLYNSGVPLQKVAKLMNHESSASTKHYLNKYNIGLDETSSEEENNNDDDDAQHNRNSSGSSGESLLYYRNE

SEQ ID NO: 22 - DNA, 1350 bp, Autographa californica
atgaacggttttaatgttcgcaacgaaaacaattttaattcttggaaaataaaaattcaatccgctccccggttcgagtccgtgttcgatttggccaccgatcggcaacgatgcacgcccgacgaggtgaaaaacaacagtctgtggagcaagtacatgttccccaaaccgtttgcgcccaccactttaaaaagttacaagtctcgattcattaaaattgtgtactgctcggtagacgatgttcacctggaagacatgtcgtactcgttggacaaggagtttgactcgatagaaaaccaaacacttctcattgatccccaagaactgtgcaggcgcatgctcgaacttcgctcggtcaccaaagaaacactacagttgactataaacttttacaccaacatgatgaacttgcccgaatacaaaattccccgcatggttatgctgccgcgcgacaaggagctcaaaaatatcagggaaaaggaaaagaatttaatgcttaaaaacgtaatagataccatattaaattttattaatgataaaattaaaatgctcaacagcgattatgttcacgaccgcggtctaattaggggcgcgatagtgttttgcatcatgttagggacgggtatgcgaatcaacgaagcgcgccaactcagcgtggacgatctcaacgtgctaattaaaagaggaaaactgcacagcgacacgattaatttaaagcgaaaacgcagtcgtaataacacactcaacaacatcaaaatgaaaccgttggaattggcacgcgagatttattcacgaaacccgaccattttgcaaatatctaaaaacacctcgacgcccttcaaagatttcaggcgactccttgaagagtcgggcgtcgagatggaacggccgcgcagcaacatgataagacattatttgagcagtaacctatacaatagcggcgtgcctttacaaaaagtggccaaattaatgaaccacgaatcctccgcaagcaccaaacattacttgaacaaatacaatataggtttagacgaaacgagcagcgaagaggagaacaacaacgacgacgacgacgcgcagcataatcgcaattcgtccggttcgtcgggagaatcgttgttgtactatcgcaacgaatagagtaagggaataaaaatgaatttatatttgttgttgggcgcactggccatatttagcctagtgtatgacaaaaaggaaaacagcatctttttgtatctgctcatattgtttctcgtgtttattatcgtcagcccggccattataagtaagaacaccgagtctaccgtagaagacataccgagtcataaagctaagagcgtccgaaaaaaa

SEQ ID NO: 23 - DNA, 267 bp, Autographa californica
ggcgctcaacgacgatactgccagcgaaccgcaacaatttagcgagcccgttcacaaaatgccaataaacgacatggtgggctatgacaacacgacgagcaacgtgtcggcgggaattataattttaattagtgtagtcgcttttatagctttattcttactgctgtatgtaatatattatttcgtaatattaagagaacaacaacaatattcggatagtattgacaccgattctccttttgtttttaataaatttgattaaccaca